(19) United States
(12) Patent Application Publication
(10) Pub. No.: US 2013/0237530 A1
(43) Pub. Date: Sep. 12, 2013

(54) BENZODIAZEPINE DERIVATIVES, COMPOSITIONS AND METHODS FOR TREATING COGNITIVE IMPAIRMENT

(71) Applicant: John A. Lowe, III, Stonington, CT (US)

(72) Inventor: John A. Lowe, III, Stonington, CT (US)

(73) Assignee: AGENEBIO, INC., Indianapolis, IN (US)

(21) Appl. No.: 13/885,561

(22) PCT Filed: Nov. 15, 2011
(86) PCT No.: PCT/US11/60840

§ 371 Date: May 15, 2013

Related U.S. Application Data
(60) Provisional application No. 61/413,975, filed on Nov. 15, 2010.

(51) Int. Cl. C07D 487/14

(52) U.S. Cl. CPC C07D 487/14
USPC 514/219; 540/555

(57) ABSTRACT

This invention relates to benzodiazepine derivatives, compositions comprising therapeutically effective amounts of those benzodiazepine derivatives and methods of using those derivatives or compositions in treating central nervous system (CNS) disorders with cognitive impairment that are responsive to agonists of α5 subunit containing GABA_{A} receptor, e.g., age-related cognitive impairment, Mild Cognitive Impairment (MCI), dementia, Alzheimer's Disease (AD), prodromal AD, post traumatic stress disorder (PTSD), schizophrenia and cancer-therapy-related cognitive impairment.

### FIELD OF THE INVENTION

This invention relates to compounds, compositions and methods for treating central nervous system (CNS) disorders with cognitive impairment that are responsive to agonists of α5 subunit containing GABA_{A} receptor, e.g., age-related cognitive impairment, Mild Cognitive Impairment (MCI), dementia, Alzheimer's Disease (AD), prodromal AD, post traumatic stress disorder (PTSD), schizophrenia and cancer-therapy-related cognitive impairment.

### BACKGROUND OF THE INVENTION

GABA_{A} receptors (GABA_{A} R) are pentameric assemblies from a pool of different subunits (α1-6, β1-3, γ1-3, δ, ε, π, θ) that forms a Cl-permeable channel that is gated by the neurotransmitter γ-aminobutyric acid (GABA). Various pharmacological effects, including anxiety disorders, epilepsy, insomnia, pre-anesthetic sedation, and muscle relaxation, are mediated by different GABA_{A} subtypes.

Various studies have demonstrated that reduced GABA signaling is linked to various CNS disorders with cognitive impairment. In particular, the α5-containing GABA_{A} Rs, which are relatively sparse in the mammalian brain, play a role in modifying learning and memory. Previous studies demonstrated a reduction of hippocampal expression of the α5 subunit of the GABA_{A} receptor in rats with age-related cognitive decline (see International Patent Publication WO 2007/019312). Such results suggest that upregulation of α5-containing GABA_{A} R function may be effective in the treatment of CNS disorders with cognitive impairment, e.g., age-related cognitive impairment, Mild Cognitive Impairment (MCI), dementia, Alzheimer's Disease (AD), prodromal AD, PTSD, schizophrenia and cancer-therapy-related cognitive impairment.

Thus, there is a need for agonists of α5-containing GABA_{A} R that are useful in therapeutic preparations for the treatment of CNS disorders with cognitive impairment.

### SUMMARY OF THE INVENTION

The present invention addresses the aforementioned need by providing compounds of formulae I:

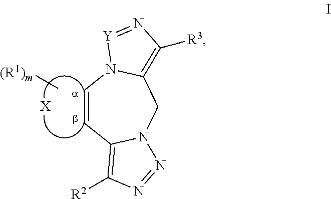

or a pharmaceutically acceptable salt thereof, wherein:
X and the two carbon atoms designated by α and β together form a C5-C10 aromatic ring having 0-4 heteroatoms independently selected from N, O and S;
Y is —N═ or —C(R⁴)═;
m is an integer selected from 0-4;
each occurrence of R¹, R², R³ and R⁴ is independently selected from:
   halogen, —R, —OR, —NO₂, —NCS, —CN, —CF₃, —OCF₃, —SiR₃, —N(R)₂, —SR, —SOR, —SO₂R, —SO₂N(R)₂, —SO₃R, —(CR₂)₁₋₃R, —(CR₂)₁₋₃—OR, —(CR₂)₀₋₃—C(O)NR(CR₂)₀₋₃R, —(CR₂)₀₋₃—C(O)NR(CR₂)₀₋₃OR, —C(O)R, —C(O)C(O)R, —C(O)CH₂C(O)R, —C(S)R, —C(S)OR, —C(O)OR, —C(O)C(O)OR, —C(O)C(O)N(R)₂, —OC(O)R, —C(O)N(R)₂, —OC(O)N(R)₂, —C(S)N(R)₂, —(CR₂)₀₋₃NHC(O)R, —N(R)N(R)COR, —N(R)N(R)C(O)OR, —N(R)N(R)CON(R)₂, —N(R)SO₂R, —N(R)SO₂N(R)₂, —N(R)C(O)OR, —N(R)C(O)R, —N(R)C(S)R, —N(R)C(O)N(R)₂, —N(R)C(S)N(R)₂, —N(COR)COR, —N(OR)R, —C(═NH)N(R)₂, —C(O)N(OR)R, —C(═NOR)R, —OP(O)(OR)₂, —P(O)(R)₂, —P(O)(OR)₂, and —P(O)(H)(OR);
each R is independently selected from:
   H—,
   (C1-C12)-aliphatic-,
   (C3-C10)-cycloalkyl-,
   (C3-C10)-cycloalkenyl-,
   [(C3-C10)-cycloalkyl]-(C1-C12)-aliphatic-,
   [(C₃-C₁₀)-cycloalkenyl]-(C1-C12)-aliphatic-,
   (C6-C10)-aryl-,
   (C6-C10)-aryl-(C1-C12)aliphatic-,
   (C3-C10)-heterocyclyl-,
   (C6-C10)-heterocyclyl-(C1-C12)aliphatic-,
   (C5-C10)-heteroaryl-, and
   (C5-C10)-heteroaryl-(C1-C12)-aliphatic-;
   or when two R groups bound to the same atom, the two R groups may be taken together with the atom to which they are bound to form a 3- to 10-membered aromatic or non-aromatic ring having 0-3 heteroatoms independently selected from N, O, S, SO, and SO₂, wherein said ring is optionally fused to a (C6-C10)aryl, (C5-C10)heteroaryl, (C3-C10)cycloalkyl, or a (C3-C10)heterocyclyl;
   wherein each occurrence of R is independently substituted with 0-5 R′;
   wherein each occurrence of R′ is independently selected from H, halogen, —R″, —OR″, —NO₂, —NCS, —CN, —CF₃, —OCF₃ and —N(R″)₂;
   wherein R″ is H or —(C1-C4)-aliphatic;
provided that said compound of formula I is not:

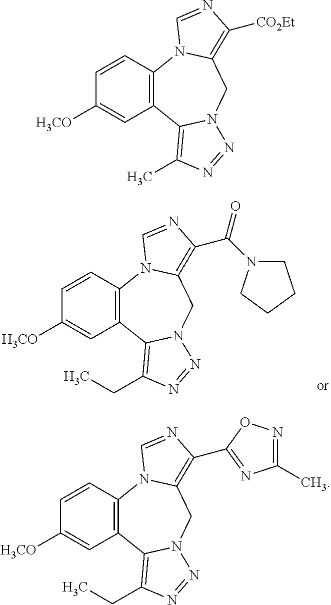

The present invention also addresses the aforementioned need by providing compounds of formulae II:

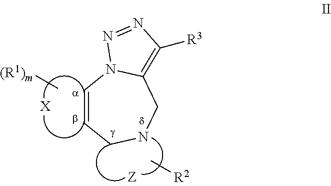

or a pharmaceutically acceptable salt thereof, wherein:
X and the two carbon atoms designated by α and β together form a C5-C10 aromatic ring having 0-4 heteroatoms independently selected from N, O and S;
Z and the carbon atom designated by γ and the N atom designated by δ together form a triazolo ring selected from:

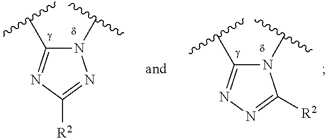

m is an integer selected from 0-4;
each occurrence of R¹, R² and R³ is independently selected from:
   halogen, —R, —OR, —NO₂, —NCS, —CN, —CF₃, —OCF₃, —SiR₃, —N(R)₂, —SR, —SOR, —SO₂R, —SO₂N(R)₂, —SO₃R, —(CR₂)₁₋₃R, —(CR₂)₁₋₃—OR, —(CR₂)₀₋₃—C(O)NR(CR₂)₀₋₃R, —(CR₂)₀₋₃—C(O)NR(CR₂)₀₋₃OR, —C(O)R, —C(O)C(O)R, —C(O)CH₂C(O)R, —C(S)R, —C(S)OR, —C(O)OR, —C(O)C(O)OR, —C(O)C(O)N(R)₂, —OC(O)R, —C(O)N(R)₂, —OC(O)N(R)₂, —C(S)N(R)₂, —(CR₂)₀₋₃NHC(O)R, —N(R)N(R)COR, —N(R)N(R)C(O)OR, —N(R)N(R)CON(R)₂, —N(R)SO₂R, —N(R)SO₂N(R)₂, —N(R)C(O)OR, —N(R)C(O)R, —N(R)C(S)R, —N(R)C(O)N(R)₂, —N(R)C(S)N(R)₂, —N(COR)COR, —N(OR)R, —C(═NH)N(R)₂, —C(O)N(OR)R, —C(═NOR)R, —OP(O)(OR)₂, —P(O)(R)₂, —P(O)(OR)₂, and —P(O)(H)(OR);
each R is independently selected from:
   H—,
   (C1-C12)-aliphatic-,
   (C3-C10)-cycloalkyl-,
   (C3-C10)-cycloalkenyl-,
   [(C3-C10)-cycloalkyl]-(C1-C12)-aliphatic-,
   [(C3-C10)-cycloalkenyl]-(C1-C12)-aliphatic-,
   (C6-C10)-aryl-,
   (C6-C10)-aryl-(C1-C12)aliphatic-,
   (C3-C10)-heterocyclyl-,
   (C6-C10)-heterocyclyl-(C1-C12)aliphatic-,
   (C5-C10)-heteroaryl-, and
   (C5-C10)-heteroaryl-(C1-C12)-aliphatic-;
   or when two R groups bound to the same atom, the two R groups may be taken together with the atom to which they are bound to form a 3- to 10-membered aromatic or non-aromatic ring having 0-3 heteroatoms independently selected from N, O, S, SO, or SO₂, wherein said ring is optionally fused to a (C6-C10)aryl, (C5-C10)heteroaryl, (C3-C10)cycloalkyl, or a (C3-C10)heterocyclyl;
   wherein each occurrence of R is independently substituted with 0-5 R′;
   wherein each occurrence of R′ is independently selected from H, halogen, —OH, —R″, —OR″, —NO₂, —NCS, —CN, —CF₃, —OCF₃ and —N(R″)₂;
   wherein R″ is H or —(C1-C4)-aliphatic.

Compounds of formulae I and II can be used to treat the conditions described herein, such as through activity as GABA_{A} α5 receptor agonists.

The present invention also provides compositions that comprise the above compounds or a pharmaceutically acceptable salt thereof.

In another aspect of the invention, there is provided a method for treating CNS disorder with cognitive impairment in a subject in need or at risk thereof, the method comprising the step of administering to said subject a therapeutically effective amount of a GABA_{A} α5 receptor agonist or a pharmaceutically acceptable salt thereof. In certain embodiments of the invention, the GABA_{A} α5 receptor agonist or a pharmaceutically acceptable salt thereof is administered every 12 or 24 hours.

### DETAILED DESCRIPTION OF THE FIGURES

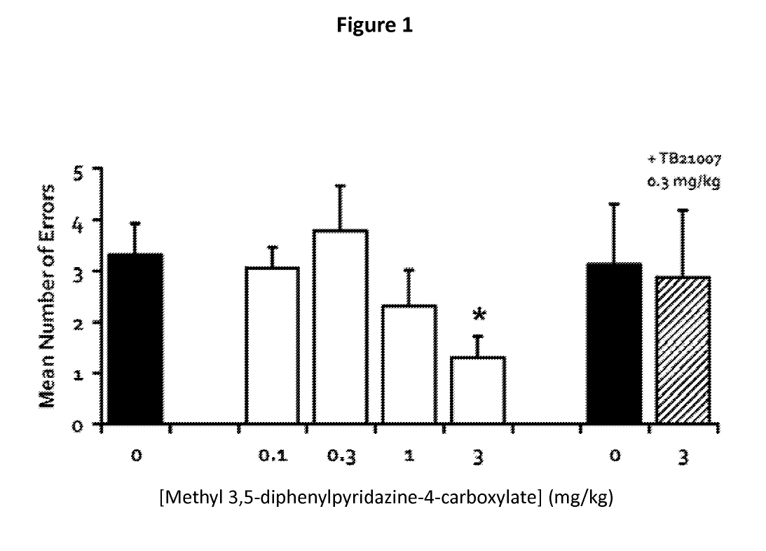

FIG. 1 is a graph depicting the effects of administering methyl 3,5-diphenylpyridazine-4-carboxylate on the spatial memory retention of ten aged-impaired (AI) rats in an eight-arm Radial Arm Maze (RAM) test. The black bars refer to rats treated with vehicle alone; open bars refer to rats treated with methyl 3,5-diphenylpyridazine-4-carboxylate at different doses; hatched bar refers to rats treated with the combination of TB21007 and methyl 3,5-diphenylpyridazine-4-carboxylate.

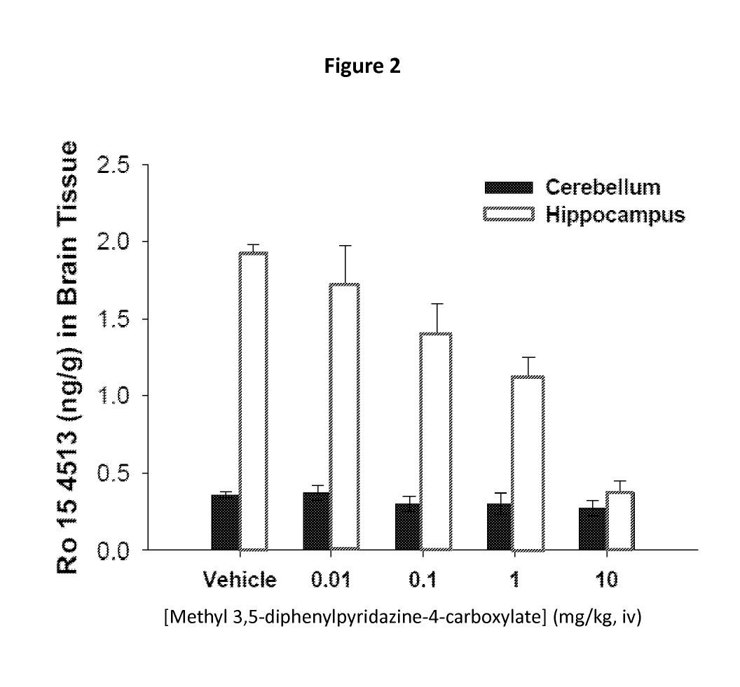

FIG. 2 is a graph showing the effect of methyl 3,5-diphenylpyridazine-4-carboxylate (administered intravenously) on the binding of Ro154513 in the hippocampus and cerebellum. Methyl 3,5-diphenylpyridazine-4-carboxylate blocked the binding of Ro154513 in the hippocampus but did not affect binding of Ro15413 in the cerebellum.

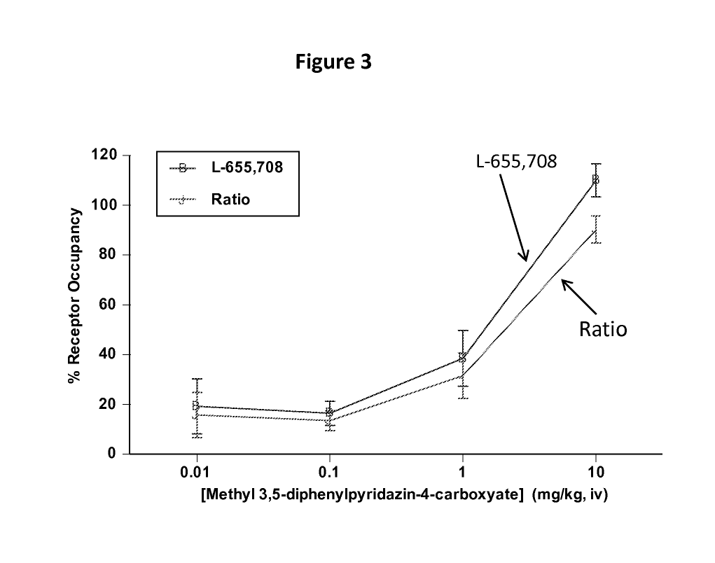

FIG. 3 is a graph showing dose-dependent GABA_{A} α5 receptor occupancy by methyl 3,5-diphenylpyridazine-4-carboxylate administered intravenously, with receptor occupancy determined either by the ratio between hippocampus (a region of high GABA_{A} α5 receptor density) exposure of RO 15-4513 and cerebellum (a region with low GABA_{A} α5 receptor density) exposure of RO 15-4513, or by using the GABA_{A} α5 selective compound L-655,708 (10 mg/kg, i.v.) to define full occupancy.

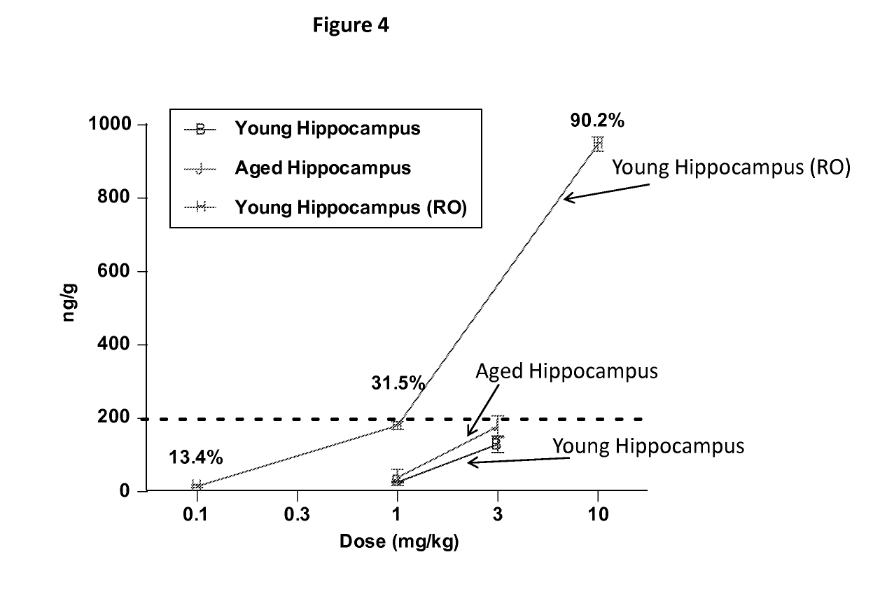

FIG. 4 is a graph showing exposure occupancy relationships for methyl 3,5-diphenylpyridazine-4-carboxylate in hippocampus. Methyl 3,5-diphenylpyridazine-4-carboxylate occupies about 32% of GABAA α5 receptors at exposures which are behaviorally active in aged-impaired rats.

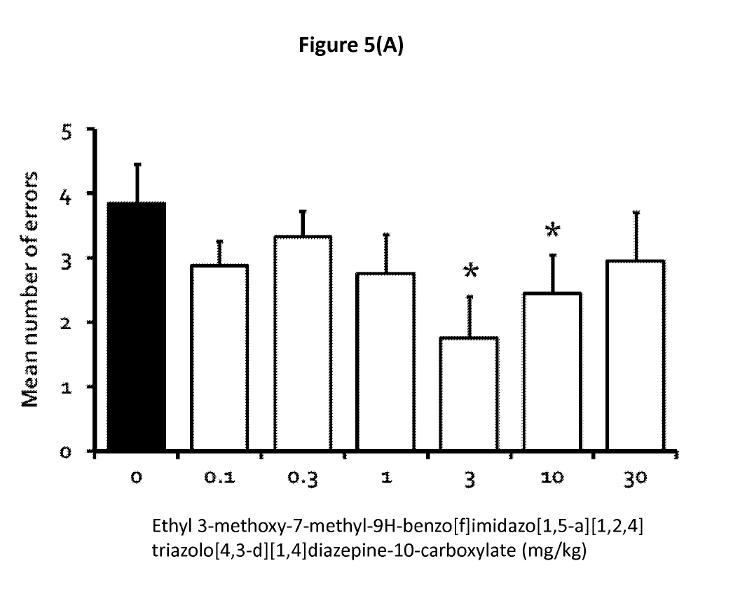

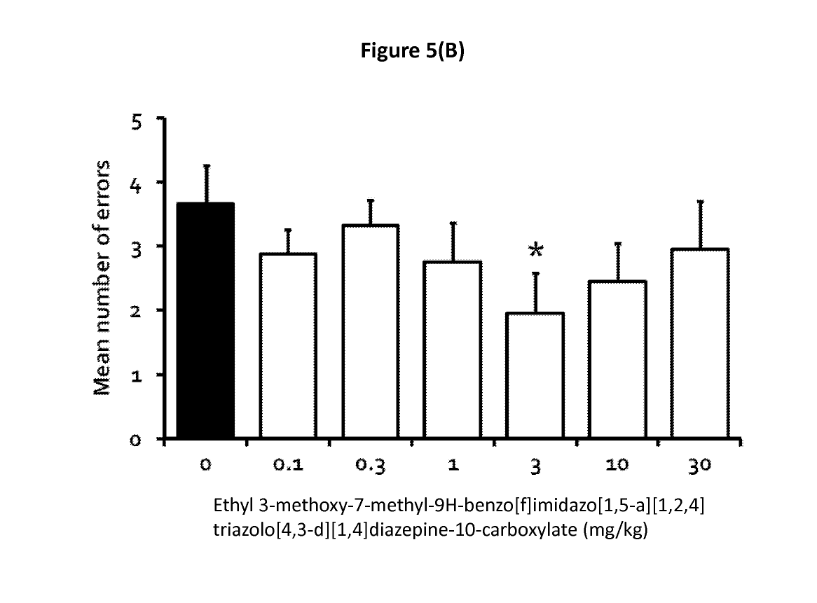

FIGS. 5(A)-(B) are graphs depicting the effect of ethyl 3-methoxy-7-methyl-9H-benzo[f]imidazo[1,5-a][1,2,4]triazolo[4,3-d][1,4]diazepine-10-carboxylate on the spatial memory retention of ten aged-impaired (AI) rats in an eight-arm Radial Arm Maze (RAM) test. FIG. 5(A) shows the effect of ethyl 3-methoxy-7-methyl-9H-benzo[f]imidazo[1,5-a][1,2,4]triazolo[4,3-d][1,4]diazepine-10-carboxylate on the spatial memory retention of ten aged-impaired (AI) rats in the RAM test, where the vehicle control was tested 3 times, and the different doses of ethyl 3-methoxy-7-methyl-9H-benzo[f]imidazo[1,5-a][1,2,4]triazolo[4,3-d][1,4]diazepine-10-carboxylate were tested twice; FIG. 5(B) shows the effect of ethyl 3-methoxy-7-methyl-9H-benzo[f]imidazo[1,5-a][1,2,4]triazolo[4,3-d][1,4]diazepine-10-carboxylate on the spatial memory retention of ten aged-impaired (AI) rats in the RAM test, where the vehicle control was tested 5 times, the 3 mg/kg dose of ethyl 3-methoxy-7-methyl-9H-benzo[f]imidazo[1,5-a][1,2,4]triazolo[4,3-d][1,4]diazepine-10-carboxylate was tested 4 times, and the other doses of ethyl 3-methoxy-7-methyl-9H-benzo[f]imidazo[1,5-a][1,2,4]triazolo[4,3-d][1,4]diazepine-10-carboxylate were tested twice. In both FIGS. 5(A) and 5(B), black bars refer to rats treated with vehicle alone and open bars refer to rats treated with ethyl 3-methoxy-7-methyl-9H-benzo[f]imidazo[1,5-a][1,2,4]triazolo[4,3-d][1,4]diazepine-10-carboxylate at different doses.

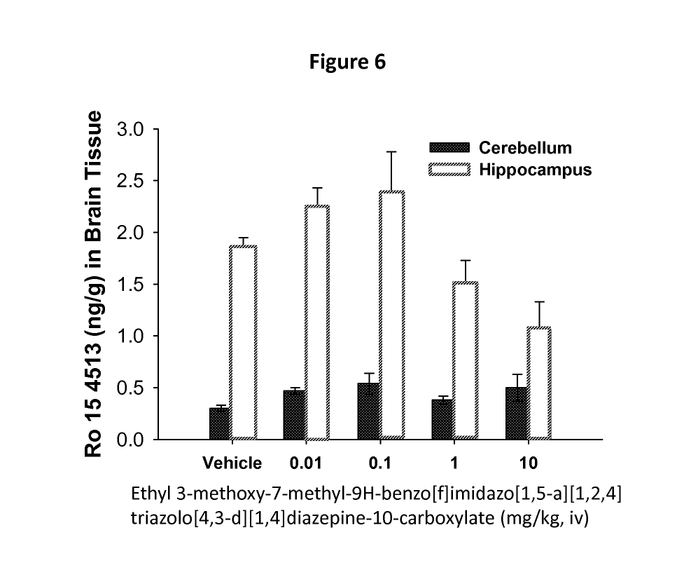

FIG. 6 is a graph showing the effect of ethyl 3-methoxy-7-methyl-9H-benzo[f]imidazo[1,5-a][1,2,4]triazolo[4,3-d][1,4]diazepine-10-carboxylate (administered intravenously) on the binding of Ro154513 in the hippocampus and cerebellum. Ethyl 3-methoxy-7-methyl-9H-benzo[f]imidazo[1,5-a][1,2,4]triazolo[4,3-d][1,4]diazepine-10-carboxylate blocked the binding of Ro154513 in the hippocampus but did not affect binding of Ro15413 in the cerebellum.

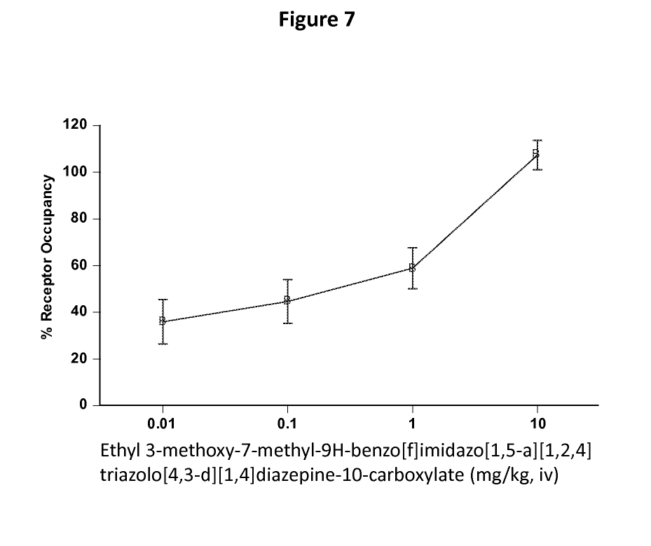

FIG. 7 is a graph showing dose-dependent GABA_{A} α5 receptor occupancy by ethyl 3-methoxy-7-methyl-9H-benzo[f]imidazo[1,5-a][1,2,4]triazolo[4,3-d][1,4]diazepine-10-carboxylate administered intravenously, as calculated by the ratio between hippocampus (a region of high GABA_{A}α5 receptor density) exposure of RO 15-4513 and cerebellum (a region with low GABA_{A}α5 receptor density) exposure of RO 15-4513 to define full occupancy.

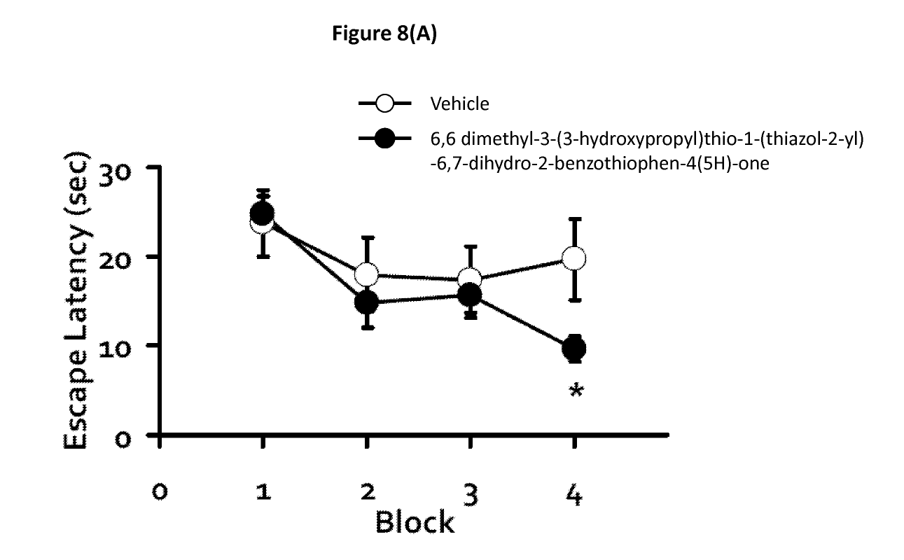

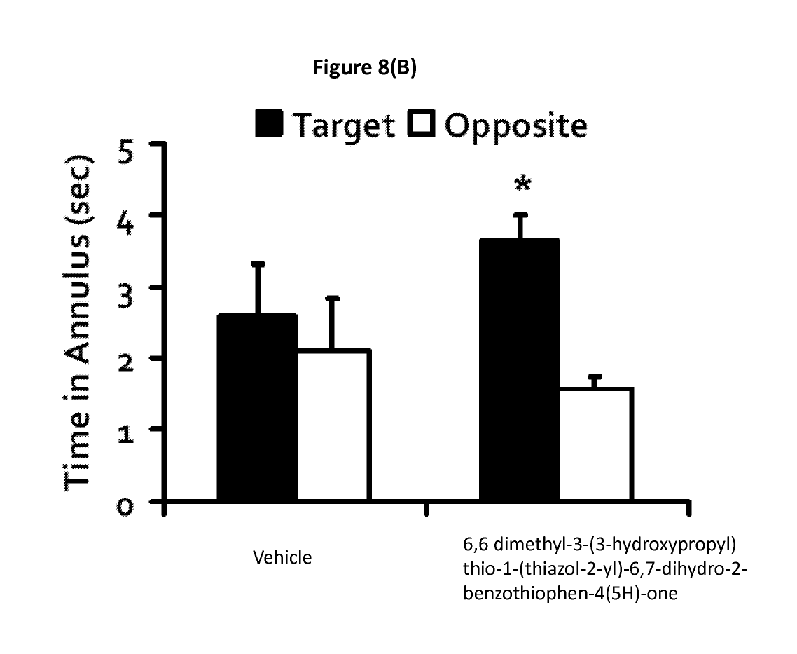

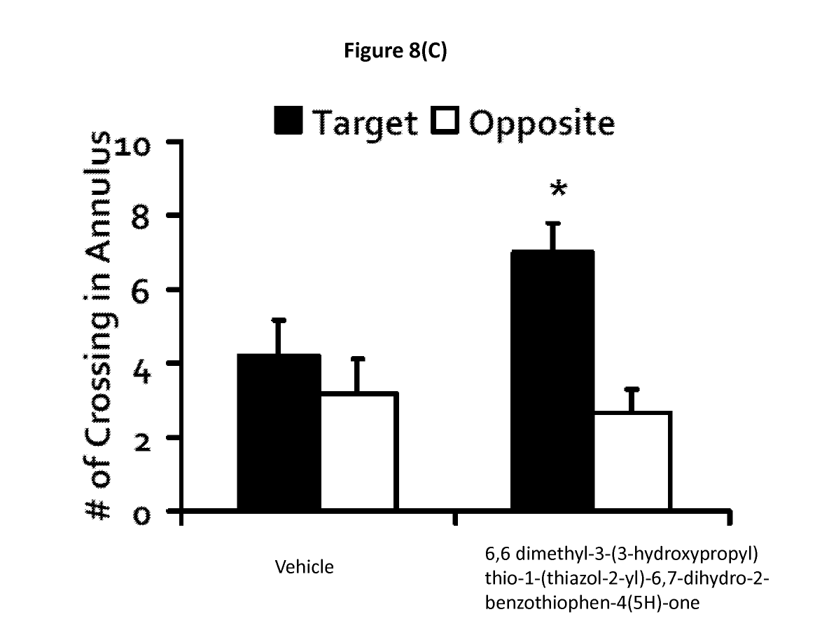

FIG. 8(A)-(C) are graphs showing the effect of 6,6 dimethyl-3-(3-hydroxypropyl)thio-1-(thiazol-2-yl)-6,7-dihydro-2-benzothiophen-4(5H)-one, as compared to vehicle dimethyl sulfoxide (DMSO), in aged-impaired rats using a Morris water maze behavioral task. FIG. 8(A) shows the escape latency (i.e., the average time in seconds rats took to find the hidden platform in the water pool) during training in rats received 6,6 dimethyl-3-(3-hydroxypropyl)thio-1-(thiazol-2-yl)-6,7-dihydro-2-benzothiophen-4(5H)-one and rats received vehicle DMSO; FIG. 8(B) shows the amount of time spent in target annulus and opposite annulus by rats received 6,6 dimethyl-3-(3-hydroxypropyl)thio-1-(thiazol-2-yl)-6,7-dihydro-2-benzothiophen-4(5H)-one and rats received vehicle DMSO; FIG. 8(C) shows number of crossing in target annulus and opposite annulus by rats received 6,6 dimethyl-3-(3-hydroxypropyl)thio-1-(thiazol-2-yl)-6,7-dihydro-2-benzothiophen-4(5H)-one and rats received vehicle DMSO.

### DETAILED DESCRIPTION OF THE INVENTION

### (1) Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well known and commonly used in the art.

The methods and techniques of the present invention are generally performed, unless otherwise indicated, according to conventional methods well known in the art and as described in various general and more specific references that are cited and discussed throughout this specification. See, e.g. “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, Mass. (2000).

Chemistry terms used herein are used according to conventional usage in the art, as exemplified by “The McGraw-Hill Dictionary of Chemical Terms”, Parker S., Ed., McGraw-Hill, San Francisco, C.A. (1985).

All of the above, and any other publications, patents and published patent applications referred to in this application are specifically incorporated by reference herein. In case of conflict, the present specification, including its specific definitions, will control.

Throughout this specification, the word “comprise” or variations such as “comprises” or “comprising” will be understood to imply the inclusion of a stated integer (or components) or group of integers (or components), but not the exclusion of any other integer (or components) or group of integers (or components).

The singular forms “a,” “an,” and “the” include the plurals unless the context clearly dictates otherwise.

The term “including” is used to mean “including but not limited to.” “Including” and “including but not limited to” are used interchangeably.

The term “agent” is used herein to denote a chemical compound (such as an organic or inorganic compound (including, such as, a compound of the present invention), a mixture of chemical compounds), a biological macromolecule (such as a nucleic acid, an antibody, including parts thereof as well as humanized, chimeric and human antibodies and monoclonal antibodies, a protein or portion thereof, e.g., a peptide, a lipid, a carbohydrate), or an extract made from biological materials such as bacteria, plants, fungi, or animal (particularly mammalian) cells or tissues. Agents include, for example, agents that are known with respect to structure, and those that are not known with respect to structure. The α5-containing GABA_{A} R agonist activity of such agents may render them suitable as “therapeutic agents” in the methods and compositions of this invention.

A “patient,” “subject,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

“Cognitive function” or “cognitive status” refers to any higher order intellectual brain process or brain state, respectively, involved in learning and/or memory including, but not limited to, attention, information acquisition, information processing, working memory, short-term memory, long-term memory, anterograde memory, retrograde memory, memory retrieval, discrimination learning, decision-making, inhibitory response control, attentional set-shifting, delayed reinforcement learning, reversal learning, the temporal integration of voluntary behavior, and expressing an interest in one's surroundings and self-care.

In humans, cognitive function may be measured, for example and without limitation, by the clinical global impression of change scale (CIBIC-plus scale); the Mini Mental State Exam (MMSE); the Neuropsychiatric Inventory (NPI); the Clinical Dementia Rating Scale (CDR); the Cambridge Neuropsychological Test Automated Battery (CANTAB) or the Sandoz Clinical Assessment-Geriatric (SCAG). See Folstein et al., J Psychiatric Res 12: 189-98, (1975); Robbins et al., Dementia 5: 266-81, (1994); Rey, L'examen clinique en psychologie, (1964); Kluger et al., J Geriatr Psychiatry Neurol 12:168-79, (1999).

In animal model systems, cognitive function may be measured in various conventional ways known in the art, including using a Morris Water Maze (MWM), Barnes circular maze, elevated radial arm maze, T maze or any other mazes in which the animals use spatial information. Other tests of cognitive function in animals include prepulse inhibition, latent inhibition, object recognitions test, delayed non-match to sample test, reaction time tasks, attentional set shifting, cross-maze set shifting task, social interaction task, and social recognition test.

Cognitive function may also be measured using imaging techniques such as Positron Emission Tomography (PET), functional magnetic resonance imaging (fMRI), Single Photon Emission Computed Tomography (SPECT), or any other imaging technique that allows one to measure brain function. In animals, cognitive function may also be measured with electrophysiological techniques.

“Promoting” cognitive function refers to affecting impaired cognitive function so that it more closely resembles the function of an aged-matched normal, unimpaired subject, or the function of a young adult subject. Cognitive function may be promoted to any detectable degree, but in humans preferably is promoted sufficiently to allow an impaired subject to carry out daily activities of normal life at the same level of proficiency as an aged-matched normal, unimpaired subject or as a young adult subject.

“Preserving” cognitive function refers to affecting normal or impaired cognitive function such that it does not decline or does not fall below that observed in the subject upon first presentation or diagnosis, or delays such decline.

“Improving” cognitive function includes promoting cognitive function and/or preserving cognitive function in a subject.

“Cognitive impairment” refers to cognitive function in subjects that is not as robust as that expected in an age-matched normal subject (i.e. subjects with mean scores for a given age in a cognitive test). In some cases, cognitive function is reduced by about 5%, about 10%, about 30%, or more, compared to cognitive function expected in an age-matched normal subject.

“Age-related cognitive impairment” refers to cognitive impairment in aged subjects, wherein their cognitive function is not as robust as that expected in an age-matched normal subject or as that expected in young adult subjects. In some cases, cognitive function is reduced by about 5%, about 10%, about 30%, or more, compared to cognitive function expected in an age-matched normal subject. In some cases, cognitive function is as expected in an age-matched normal subject, but reduced by about 5%, about 10%, about 30%, about 50% or more, compared to cognitive function expected in a young adult subject. Age-related impaired cognitive function may be associated with Mild Cognitive Impairment (MCI) (including amnestic MCI and non-amnestic MCI), Age-Associated Memory Impairment (AAMI), and Age-related Cognitive Decline (ARCD).

“Mild Cognitive Impairment” or “MCI” refers to a condition characterized by low-level cognitive deficit causing no problems in normal activities of daily living. A clinical characterization of MCI may comprise: presence of a cognitive complaint in at least one cognitive domain expressed by subject or informant, objective evidence of impairment on neuropsychological testing of at least 1.5 standard deviations below norms matched for age, and activities of daily living remaining intact. The cognitive deficit in subjects with MCI may involve any cognition area or mental process including memory, language, association, attention, perception, problem solving, executive function and visuospatial skills. See, e.g., Winbald et al., J. Intern. Med. 256:240-240, 2004; Meguro, Acta. Neural. Taiwan. 15:55-57, 2008; Ellison et al., CNS Spectr. 13:66-72, 2008, Petersen, Semin. Neural. 27:22-31, 2007. MCI is further subdivided into amnestic MCI (aMCI) and non-amnestic MCI, characterized by the impairment (or lack thereof) of memory in particular. MCI is defined as aMCI if memory is found to be impaired given the age and education level of the subject. If, on the other hand, the memory of the subject is found to be intact for age and education, but other non-memory cognitive domains are impaired, such as language, executive function, or visuospatial skills, MCI is defines an non-amnestic MCI. aMCI and non-amnestic MCI can both be further subdivided into single or multiple domain MCI. aMCI-single domain refers to a condition where memory, but not other cognitive areas are impaired. aMCI-multiple domain refers to a condition where memory and at least one other cognitive area are impaired. Non-amnestic MCI is single domain or multiple domain dependent on whether nor not more than one non-memory cognitive area is impaired. See, e.g., Peterson and Negash, CNS Spectr. 13:45-53, 2008.

“Age-Associate Memory Impairment (AAMI)” refers to a decline in memory due to aging. A patient may be considered to have AAMI if he or she is at least 50 years old and meets all of the following criteria: a) the patient has noticed a decline in memory performance, b) the patient performs worse on a standard test of memory compared to young adults, and c) all other obvious causes of memory decline, except normal aging, have been ruled out (in other words, the memory decline cannot be attributed to other causes such as a recent heart attack or head injury, depression, adverse reactions to medication, Alzheimer's disease, etc.).

“Age-Related Cognitive Decline (ARCD)” refers to declines in memory and cognitive abilities that are a normal consequence of aging in humans (e.g., Craik & Salthouse, 1992). This is also true in virtually all mammalian species. Age-Associated Memory Impairment refers to older persons with objective memory declines relative to their younger years, but cognitive functioning that is normal relative to their age peers (Crook et al., 1986). Age-Consistent Memory Decline, is a less pejorative label which emphasizes that these are normal developmental changes (Crook, 1993; Larrabee, 1996), are not pathophysiological (Smith et al., 1991), and rarely progress to overt dementia (Youngjohn & Crook, 1993). The DSM-IV (1994) has codified the diagnostic classification of ARCD.

“Dementia” refers to a condition characterized by severe cognitive deficit that interferes in normal activities of daily living. Subjects with dementia also display other symptoms such as impaired judgment, changes in personality, disorientation, confusion, behavior changes, trouble speaking, and motor deficits. There are different types of dementias, such as Alzheimer's disease (AD), vascular dementia, dementia with Lewy bodies, and frontotemporal dementia.

Alzheimer's disease (AD) is characterized by memory deficits in its early phase. Later symptoms include impaired judgment, disorientation, confusion, behavior changes, trouble speaking, and motor deficits. Histologically, AD is characterized by beta-amyloid plaques and tangles of protein tau.

Vascular dementia is caused by strokes. Symptoms overlap with those of AD, but without the focus on memory impairment.

Dementia with Lewy bodies is characterized by abnormal deposits of alpha-synuclein that form inside neurons in the brain. Cognitive impairment may be similar to AD, including impairments in memory and judgment and behavior changes.

Frontotemporal dementia is characterized by gliosis, neuronal loss, superficial spongiform degeneration in the frontal cortex and/or anterior temporal lobes, and Picks' bodies. Symptoms include changes in personality and behavior, including a decline in social skills and language expression/comprehension.

“Post traumatic stress disorder (PTSD)” refers to an anxiety disorder characterized by an immediate or delayed response to a catastrophic event, characterized by re-experiencing the trauma, psychic numbing or avoidance of stimuli associated with the trauma, and increased arousal. Re-experiencing phenomena include intrusive memories, flashbacks, nightmares, and psychological or physiological distress in response to trauma reminders. Such responses produce anxiety and can have significant impact, both chronic and acute, on a patient's quality of life and physical and emotional health. PTSD is also associated with impaired cognitive performance, and older individuals with PTSD have greater decline in cognitive performance relative to control patients.

“Schizophrenia” refers to a chronic debilitating disorder, characterized by a spectrum of psychopathology, including positive symptoms such as aberrant or distorted mental representations (e.g., hallucinations, delusions), negative symptoms characterized by diminution of motivation and adaptive goal-directed action (e.g., anhedonia, affective flattening, avolition), and cognitive impairment. While abnormalities in the brain are proposed to underlie the full spectrum of psychopathology in schizophrenia, currently available antipsychotics are largely ineffective in treating cognitive impairments in patients.

“Cancer therapy-related cognitive impairment” refers to cognitive impairment that develops in subjects that are treated with cancer therapies such as chemotherapy and radiation. Cytotoxicity and other adverse side-effects on the brain of cancer therapies result in cognitive impairment in such functions as memory, learning and attention.

“Treating” a condition or patient refers to taking steps to obtain beneficial or desired results, including clinical results. Beneficial or desired clinical results include, but are not limited to, alleviation, amelioration, or slowing the progression, of one or more symptoms associated with age-related cognitive impairment, Mild Cognitive Impairment (MCI), dementia, Alzheimer's Disease (AD), prodromal AD, PTSD, schizophrenia and cancer therapy-related cognitive impairment.

“Treating cognitive impairment” refers to taking steps to improve cognitive function in a subject with cognitive impairment so that the subject's performance in one or more cognitive tests is improved to any detectable degree, or is prevented from further decline. Preferably, that subject's cognitive function, after treatment of cognitive impairment, more closely resembles the function of an aged-matched normal, unimpaired subject, or the function of a young adult subject. Treatment of cognitive impairment in humans may improve cognitive function to any detectable degree, but is preferably improved sufficiently to allow the impaired subject to carry out daily activities of normal life at the same level of proficiency as an aged-matched normal, unimpaired subject or as a young adult subject.

“Administering” or “administration of” a substance, a compound or an agent to a subject can be carried out using one of a variety of methods known to those skilled in the art. For example, a compound or an agent can be administered, intravenously, arterially, intradermally, intramuscularly, intraperitonealy, intravenously, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods. In some aspects, the administration includes both direct administration, including self-administration, and indirect administration, including the act of prescribing a drug. For example, as used herein, a physician who instructs a patient to self-administer a drug, or to have the drug administered by another and/or who provides a patient with a prescription for a drug is administering the drug to the patient.

Appropriate methods of administering a substance, a compound or an agent to a subject will also depend, for example, on the age of the subject, whether the subject is active or inactive at the time of administering, whether the subject is cognitively impaired at the time of administering, the extent of the impairment, and the chemical and biological properties of the compound or agent (e.g. solubility, digestibility, bioavailability, stability and toxicity). Preferably, a compound or an agent is administered orally, e.g., to a subject by ingestion. In some embodiments, the orally administered compound or agent is in an extended release or slow release formulation, or administered using a device for such slow or extended release.

A “therapeutically effective amount” or a “therapeutically effective dose” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, the nature and extent of cognitive impairment or other symptoms of the CNS disorder (such as age-related cognitive impairment, Mild Cognitive Impairment (MCI), dementia, Alzheimer's Disease (AD), prodromal AD, PTSD, schizophrenia and cancer therapy-related cognitive impairment), and the therapeutics or combination of therapeutics selected for administration, and the mode of administration. The skilled worker can readily determine the effective amount for a given situation by routine experimentation.

The compounds of the present invention also include prodrugs, analogs or derivatives. The term “prodrug” is art-recognized and is intended to encompass compounds or agents which, under physiological conditions, are converted into α5-containing GABA_{A} R agonist. A common method for making a prodrug is to select moieties which are hydrolyzed or metabolized under physiological conditions to provide the desired compound or agent. In other embodiments, the prodrug is converted by an enzymatic activity of the host animal to a GABA_{A} α5 receptor agonist.

An “α5-containing GABA_{A} R agonist” or a “GABA_{A} α5 receptor agonist” as used herein refer to a compound that up-regulates the function of α5-containing GABA_{A} R, i.e., a compound that increases GABA-gated Cl⁻ currents. In some embodiments, α5-containing GABA_{A} R agonist as used herein refers to a positive allosteric modulator, which potentiates the activity of GABA.

“Analog” is used herein to refer to a compound which functionally resembles another chemical entity, but does not share the identical chemical structure. For example, an analog is sufficiently similar to a base or parent compound such that it can substitute for the base compound in therapeutic applications, despite minor structural differences. i.e., be a GABA_{A} α5 receptor agonist.

“Derivative” is used herein to refer to the chemical modification of a compound. Chemical modifications of a compound can include, for example, replacement of hydrogen by an alkyl, acyl, or amino group. Many other modifications are also possible.

The term “aliphatic” as used herein means a straight chained or branched alkyl, alkenyl or alkynyl. It is understood that alkenyl or alkynyl embodiments need at least two carbon atoms in the aliphatic chain. Aliphatic groups typically contains from 1 (or 2) to 12 carbons, such as from 1 (or 2) to 4 carbons.

The term “aryl” as used herein means a monocyclic or bicyclic carbocyclic aromatic ring system. For example, aryl as used herein can be a C5-C10 monocyclic or C8-C12 bicyclic carbocyclic aromatic ring system. Phenyl is an example of a monocyclic aromatic ring system. Bicyclic aromatic ring systems include systems wherein both rings are aromatic, e.g., naphthyl, and systems wherein only one of the two rings is aromatic, e.g., tetralin.

The term “heterocyclic” as used herein means a monocyclic or bicyclic non-aromatic ring system having 1 to 3 heteroatom or heteroatom groups in each ring selected from O, N, NH, S, SO, or SO₂ in a chemically stable arrangement. For example, heterocyclic as used herein can be a C5-C10 monocyclic or C8-C12 bicyclic non-aromatic ring system having 1 to 3 heteroatom or heteroatom groups in each ring selected from O, N, NH, S, SO, or SO₂ in a chemically stable arrangement. In a bicyclic non-aromatic ring system embodiment of “heterocyclyl”, one or both rings may contain said heteroatom or heteroatom groups. In another bicyclic embodiment of “heterocyclic”, one of the two rings is aromatic. In another heterocyclic ring system embodiment, a non-aromatic heterocyclic ring may optionally be fused to an aromatic carbocycle.

Examples of heterocyclic rings include 3-1H-benzimidazol-2-one, 3-(1-alkyl)-benzimidazol-2-one, 2-tetrahydrofuranyl, 3-tetrahydrofuranyl, 2-tetrahydrothiophenyl, 3-tetrahydrothiophenyl, 2-morpholino, 3-morpholino, 4-morpholino, 2-thiomorpholino, 3-thiomorpholino, 4-thiomorpholino, 1-pyrrolidinyl, 2-pyrrolidinyl, 3-pyrrolidinyl, 1-tetrahydropiperazinyl, 2-tetrahydropiperazinyl, 3-tetrahydropiperazinyl, 1-piperidinyl, 2-piperidinyl, 3-piperidinyl, 1-pyrazolinyl, 3-pyrazolinyl, 4-pyrazolinyl, 5-pyrazolinyl, 1-piperidinyl, 2-piperidinyl, 3-piperidinyl, 4-piperidinyl, 2-thiazolidinyl, 3-thiazolidinyl, 4-thiazolidinyl, 1-imidazolidinyl, 2-imidazolidinyl, 4-imidazolidinyl, 5-imidazolidinyl, indolinyl, tetrahydroquinolinyl, tetrahydroisoquinolinyl, benzothiolane, benzodithiane, and 1,3-dihydro-imidazol-2-one.

The term “heteroaryl” as used herein means a monocyclic or bicyclic aromatic ring system having 1 to 3 heteroatom or heteroatom groups in each ring selected from O, N, NH or S in a chemically stable arrangement. For example, heteroaryl as used herein can be a C5-C10 monocyclic or C8-C12 bicyclic aromatic ring system having 1 to 3 heteroatom or heteroatom groups in each ring selected from O, N, NH or S in a chemically stable arrangement. In such a bicyclic aromatic ring system embodiment of “heteroaryl”:
both rings are aromatic; and
one or both rings may contain said heteroatom or heteroatom groups.

Examples of heteroaryl rings include 2-furanyl, 3-furanyl, N-imidazolyl, 2-imidazolyl, 4-imidazolyl, 5-imidazolyl, benzimidazolyl, 3-isoxazolyl, 4-isoxazolyl, 5-isoxazolyl, 2-oxazolyl, 4-oxazolyl, 5-oxazolyl, N-pyrrolyl, 2-pyrrolyl, 3-pyrrolyl, 2-pyridyl, 3-pyridyl, 4-pyridyl, 2-pyrimidinyl, 4-pyrimidinyl, 5-pyrimidinyl, pyridazinyl (e.g., 3-pyridazinyl), 2-thiazolyl, 4-thiazolyl, 5-thiazolyl, tetrazolyl (e.g., 5-tetrazolyl), triazolyl (e.g., 2-triazolyl and 5-triazolyl), 2-thienyl, 3-thienyl, benzofuryl, benzothiophenyl, indolyl (e.g., 2-indolyl), pyrazolyl (e.g., 2-pyrazolyl), isothiazolyl, 1,2,3-oxadiazolyl, 1,2,5-oxadiazolyl, 1,2,4-oxadiazolyl, 1,2,3-triazolyl, 1,2,3-thiadiazolyl, 1,3,4-thiadiazolyl, 1,2,5-thiadiazolyl, purinyl, pyrazinyl, 1,3,5-triazinyl, quinolinyl (e.g., 2-quinolinyl, 3-quinolinyl, 4-quinolinyl), and isoquinolinyl (e.g., 1-isoquinolinyl, 3-isoquinolinyl, or 4-isoquinolinyl).

The term “cycloalkyl or cycloalkenyl” refers to a monocyclic or fused or bridged bicyclic carbocyclic ring system that is not aromatic. For example, cycloalkyl or cycloalkenyl as used herein can be a C5-C10 monocyclic or fused or bridged C8-C12 bicyclic carbocyclic ring system that is not aromatic. Cycloalkenyl rings have one or more units of unsaturation. Preferred cycloalkyl or cycloalkenyl groups include cyclopropyl, cyclobutyl, cyclopentyl, cyclohexyl, cyclohexenyl, cycloheptyl, cycloheptenyl, norbornyl, adamantyl and decalinyl.

As used herein, the carbon atom designations may have the indicated integer and any intervening integer. For example, the number of carbon atoms in a (C1-C4)-alkyl group is 1, 2, 3, or 4. It should be understood that these designation refer to the total number of atoms in the appropriate group. For example, in a (C3-C10)-heterocyclyl the total number of carbon atoms and heteroatoms is 3 (as in aziridine), 4, 5, 6 (as in morpholine), 7, 8, 9, or 10.

“Pharmaceutically acceptable salts” is used herein to refer to an agent or a compound according to the invention that is a therapeutically active, non-toxic base and acid salt form of the compounds. The acid addition salt form of a compound that occurs in its free form as a base can be obtained by treating said free base form with an appropriate acid such as an inorganic acid, for example, a hydrohalic such as hydrochloric or hydrobromic, sulfuric, nitric, phosphoric and the like; or an organic acid, such as, for example, acetic, hydroxyacetic, propanoic, lactic, pyruvic, malonic, succinic, maleic, fumaric, malic, tartaric, citric, methanesulfonic, ethanesulfonic, benzenesulfonic, p-toluenesulfonic, cyclic, salicylic, p-aminosalicylic, pamoic and the like. See, e.g., WO 01/062726.

Compounds containing acidic protons may be converted into their therapeutically active, non-toxic base addition salt form, e.g. metal or amine salts, by treatment with appropriate organic and inorganic bases. Appropriate base salt forms include, for example, ammonium salts, alkali and earth alkaline metal salts, e.g., lithium, sodium, potassium, magnesium, calcium salts and the like, salts with organic bases, e.g. N-methyl-D-glucamine, hydrabamine salts, and salts with amino acids such as, for example, arginine, lysine and the like. Conversely, said salt forms can be converted into the free forms by treatment with an appropriate base or acid. Compounds and their salts can be in the form of a solvate, which is included within the scope of the present invention. Such solvates include for example hydrates, alcoholates and the like. See, e.g., WO 01/062726.

Many of the compounds useful in the methods and compositions of this invention have at least one stereogenic center in their structure. This stereogenic center may be present in a R or a S configuration, said R and S notation is used in correspondence with the rules described in Pure Appl. Chem. (1976), 45, 11-30. The invention also relates to all stereoisomeric forms such as enantiomeric and diastereoisomeric forms of the compounds or mixtures thereof (including all possible mixtures of stereoisomers). See, e.g., WO 01/062726.

Furthermore, certain compounds which contain alkenyl groups may exist as Z (zusammen) or E (entgegen) isomers. In each instance, the invention includes both mixture and separate individual isomers. Multiple substituents on a piperidinyl or the azepanyl ring can also stand in either cis or trans relationship to each other with respect to the plane of the piperidinyl or the azepanyl ring. Some of the compounds may also exist in tautomeric forms. Such forms, although not explicitly indicated in the formulae described herein, are intended to be included within the scope of the present invention. With respect to the methods and compositions of the present invention, reference to a compound or compounds is intended to encompass that compound in each of its possible isomeric forms and mixtures thereof unless the particular isomeric form is referred to specifically. See, e.g., WO 01/062726.

The invention provides compounds that upregulate the function of α5-containing GABA_{A} R, i.e., α5-containing GABA_{A} R agonists (or positive allosteric modulators) that increase GABA-gated Cl⁻ currents.

The invention further provides pharmaceutical compositions comprising one or more compounds of the invention together with a pharmaceutically acceptable carrier or excipient.

The invention further provides methods for treating CNS disorders with cognitive impairment that are responsive to agonists of α5-containing GABA_{A} receptor, e.g., age-related cognitive impairment, MCI, dementia, AD, prodromal AD, PTSD, schizophrenia and cancer therapy-related cognitive impairment. In certain embodiments, the method is a method of treating the cognitive impairment associated with age-related cognitive impairment, MCI, dementia, AD, prodromal AD, PTSD, schizophrenia and cancer therapy-related cognitive impairment.

The various CNS disorders with cognitive impairment (e.g., age-related cognitive impairment, MCI, dementia, AD, prodromal AD, PTSD, schizophrenia and cancer therapy-related cognitive impairment) may have a variety of etiologies. However, the symptom of cognitive impairment in each of the above-mentioned disorders may have overlapping causes. Thus, a composition or method of treatment that treats cognitive impairment in one CNS disorder may also treat cognitive impairment in another.

### (2) Benzodiazepine Derivatives and Compositions

The present invention provides a compound of formula I:

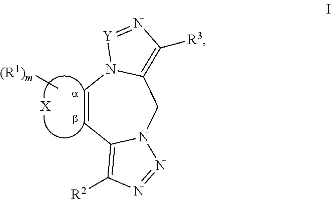

or a pharmaceutically acceptable salt thereof, wherein:
X and the two carbon atoms designated by α and β together form a C5-C10 aromatic ring having 0-4 heteroatoms independently selected from N, O and S;
Y is —N═ or —C(R⁴)═;
m is an integer selected from 0-4;
each occurrence of R¹, R², R³ and R⁴ is independently selected from:
   halogen, —R, —OR, —NO₂, —NCS, —CN, —CF₃, —OCF₃, —SiR₃, —N(R)₂, —SR, —SOR, —SO₂R, —SO₂N(R)₂, —SO₃R, —(CR₂)₁₋₃R, —(CR₂)₁₋₃—OR, —(CR₂)₀₋₃—C(O)NR(CR₂)₀₋₃R, —(CR₂)₀₋₃—C(O)NR(CR₂)₀₋₃OR, —C(O)R, —C(O)C(O)R, —C(O)CH₂C(O)R, —C(S)R, —C(S)OR, —C(O)OR, —C(O)C(O)OR, —C(O)C(O)N(R)₂, —OC(O)R, —C(O)N(R)₂, —OC(O)N(R)₂, —C(S)N(R)₂, —(CR₂)₀₋₃NHC(O)R, —N(R)N(R)COR, —N(R)N(R)C(O)OR, —N(R)N(R)CON(R)₂, —N(R)SO₂R, —N(R)SO₂N(R)₂, —N(R)C(O)OR, —N(R)C(O)R, —N(R)C(S)R, —N(R)C(O)N(R)₂, —N(R)C(S)N(R)₂, —N(COR)COR, —N(OR)R, —C(═NH)N(R)₂, —C(O)N(OR)R, —C(═NOR)R, —OP(O)(OR)₂, —P(O)(R)₂, —P(O)(OR)₂, and —P(O)(H)(OR);
each R is independently selected from:
   H—,
   (C1-C12)-aliphatic-,
   (C3-C10)-cycloalkyl-,
   (C3-C10)-cycloalkenyl-,
   (C3-C10)-cycloalkyl-(C1-C12)-aliphatic-,
   (C3-C10)-cycloalkenyl-(C1-C12)-aliphatic-,
   (C6-C10)-aryl-,
   (C6-C10)-aryl-(C1-C12)aliphatic-,
   (C3-C10)-heterocyclyl-,
   (C6-C10)-heterocyclyl-(C1-C12)aliphatic-,
   (C5-C10)-hetero aryl-, and
   (C5-C10)-heteroaryl-(C1-C12)-aliphatic-;
   or when two R groups are bound to the same atom, the two R groups may be taken together with the atom to which they are bound to form a 3- to 10-membered aromatic or non-aromatic ring having 0-3 heteroatoms independently selected from N, O, S, SO, or SO₂, wherein said ring is optionally fused to a (C6-C10)aryl, (C5-C10)heteroaryl, (C3-C10)cycloalkyl, or a (C3-C10)heterocyclyl;
   wherein each occurrence of R is independently substituted with 0-5 R′;
   wherein each occurrence of R′ is independently selected from —H, halogen, —R″, —OR″, —NO₂, —NCS, —CN, —CF₃, —OCF₃ and —N(R″)₂;
   wherein R″ is —H or —(C1-C4)-aliphatic.

In certain embodiments, the compound of the present invention is not:

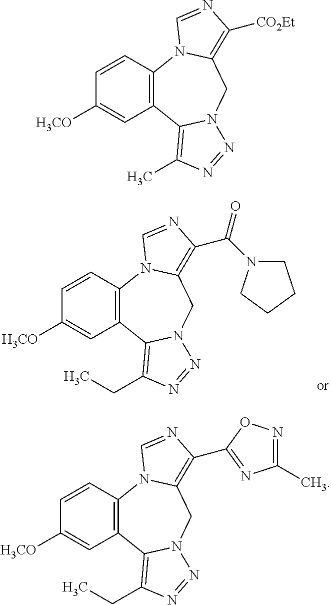

The present invention also provides a compound of formula II:

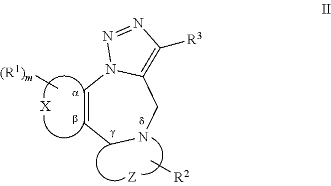

or a pharmaceutically acceptable salt thereof, wherein:
X and the two carbon atoms designated by α and β together form a C5-C10 aromatic ring having 0-4 heteroatoms independently selected from N, O and S;
Z and the carbon atom designated by γ and the nitrogen atom designated by δ together form a triazolo ring selected from:

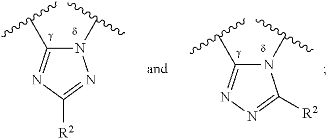

m is an integer selected from 0-4;
each occurrence of R¹, R² and R³ is independently selected from:
   halogen, —R, —OR, —NO₂, —NCS, —CN, —CF₃, —OCF₃, —SiR₃, —N(R)₂, —SR, —SOR, —SO₂R, —SO₂N(R)₂, —SO₃R, —(CR₂)₁₋₃R, —(CR₂)₁₋₃—OR, —(CR₂)₀₋₃—C(O)NR(CR₂)₀₋₃R, —(CR₂)₀₋₃—C(O)NR(CR₂)₀₋₃OR, —C(O)R, —C(O)C(O)R, —C(O)CH₂C(O)R, —C(S)R, —C(S)OR, —C(O)OR, —C(O)C(O)OR, —C(O)C(O)N(R)₂, —OC(O)R, —C(O)N(R)₂, —OC(O)N(R)₂, —C(S)N(R)₂, —(CR₂)₀₋₃NHC(O)R, —N(R)N(R)COR, —N(R)N(R)C(O)OR, —N(R)N(R)CON(R)₂, —N(R)SO₂R, —N(R)SO₂N(R)₂, —N(R)C(O)OR, —N(R)C(O)R, —N(R)C(S)R, —N(R)C(O)N(R)₂, —N(R)C(S)N(R)₂, —N(COR)COR, —N(OR)R, —C(═NH)N(R)₂, —C(O)N(OR)R, —C(═NOR)R, —OP(O)(OR)₂, —P(O)(R)₂, —P(O)(OR)₂, and —P(O)(H)(OR);
each R is independently selected from:
   H—,
   (C1-C12)-aliphatic-,
   (C3-C10)-cycloalkyl-,
   (C3-C10)-cycloalkenyl-,
   (C3-C10)-cycloalkyl-(C1-C12)-aliphatic-,
   (C3-C10)-cycloalkenyl-(C1-C12)-aliphatic-,
   (C6-C10)-aryl-,
   (C6-C10)-aryl-(C1-C12)aliphatic-,
   (C3-C10)-heterocyclyl-,
   (C6-C10)-heterocyclyl-(C1-C12)aliphatic-,
   (C5-C10)-heteroaryl-, and
   (C5-C10)-heteroaryl-(C1-C12)-aliphatic-;
   or when two R groups are bound to the same atom, the two R groups may be taken together with the atom to which they are bound to form a 3- to 10-membered aromatic or non-aromatic ring having 0-3 heteroatoms independently selected from N, O, S, SO, or SO₂, wherein said ring is optionally fused to a (C6-C10)aryl, (C5-C10)heteroaryl, (C3-C10)cycloalkyl, or a (C3-C10)heterocyclyl;
   wherein each occurrence of R is independently substituted with 0-5 R′;
   wherein each occurrence of R′ is independently selected from H, halogen, —OH, —R″, —OR″, —NO₂, —NCS, —CN, —CF₃, —OCF₃ and —N(R″)₂;
   wherein R″ is H or —(C1-C4)-aliphatic.

In certain embodiments for a compound of formula I, Y is —C(R⁴)═. For example, Y can be —CH═.

In some embodiments for a compound of formula I or II, X and the two carbon atoms designated by α and β together form a phenyl ring, optionally substituted with m occurrences of R¹. In some embodiments, m is 1.

In certain embodiments, the compound of the invention has a formula of I-A or II-A:

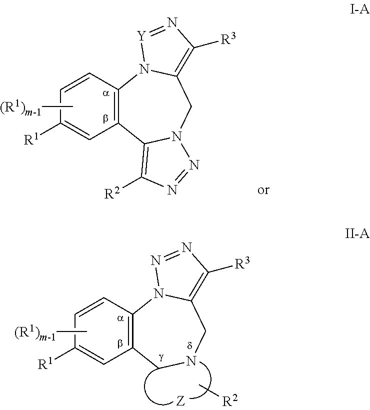

wherein all the variables can be as defined in any of the embodiments herein.

The following description applies to any of the embodiments of Formulae I, I-A, II and II-A as described herein.

According to some embodiments, the present invention provides a compound, wherein m is an integer selected from 1-4 and at least one R¹ is —OR, wherein R is (C1-C12)-aliphatic-, such as (C1-C12)-alkyl-, substituted with 0-5 R′. In some embodiments, m is an integer selected from 1-4 and at least one R¹ is —OR, wherein R is unsubstituted (C1-C4)-aliphatic-, such as methyl. In certain embodiments, one R¹ is present.

In some embodiments, the present invention provides a compound, wherein m is an integer selected from 1-4 and at least one R¹ is (C1-C12)-aliphatic-, such as (C1-C12)-alkyl-, substituted with 0-5 R′. In certain embodiments, at least one R¹ is substituted with at least one —OH. In other embodiments, m is an integer selected from 1-4 and at least one R¹ is halogen, such as Cl— or Br—. In certain of these embodiments, one R¹ is present.

The present invention also provides a compound, wherein R² is (C1-C12)-aliphatic- substituted with 0-5 R′. In some embodiments, R² is (C1-C4)-aliphatic-, such as (C1-C4)-alkyl-. In some embodiments, R² is methyl, ethyl or isopropyl.

According to certain embodiments, the present invention also provides a compound, wherein R³ is (C1-C12)-aliphatic-substituted with 0-5 R′. In some embodiments, R³ is (C1-C4)-aliphatic-, such as (C1-C4)-alkyl-. In another embodiment, R³ is substituted with at least one halogen. In some embodiments, R³ is difluoromethyl.

In another aspect, the present invention provides a compound, wherein R³ is —C(O)OR, wherein the R is (C1-C12)-aliphatic-substituted with 0-5 R′. In some embodiments, R³ is —C(O)OR, wherein R is (C1-C4)-aliphatic-, such as (C1-C4)-alkyl- and particularly methyl or ethyl.

According to some embodiments of the invention, R³ is —C(O)N(R)₂. In a specific embodiment, R³ is —C(O)N(R)₂ wherein at least one occurrence of R is —H. In another embodiment, R³ is —C(O)N(R)₂, wherein each R is independently (C1-C4)-aliphatic-, such as (C1-C4)-alkyl-. In some embodiments, R³ is —C(O)N(R)₂, wherein each R is independently methyl or ethyl. In yet another embodiment, R³ is —C(O)N(R)₂, wherein the two R groups together with the nitrogen atom to which they are bound optionally form a 3- to 10-membered aromatic or non-aromatic ring having 0-3 additional heteroatoms independently selected from N, O, S, SO, and SO₂. In a more specific embodiment, R³ is —C(O)N(R)₂, wherein the two R groups together with the nitrogen atom to which they are bound optionally form a 5- or 6-membered aromatic or non-aromatic ring having 0-3 additional heteroatoms independently selected from N, O, S, SO, and SO₂.

The present invention also provides a compound, wherein R³ is (C5-C10)-heteroaryl-, optionally substituted with at least one (C1-C4)-aliphatic-, such as (C1-C4)-alkyl-. Examples of suitable heteroaryl include 5- and 6-membered heteroaryls, particularly those containing at least one nitrogen atom and at least one oxygen atom, such as where an oxygen and a nitrogen atom are in the ring each one position away from where R³ connects to the rest of the structure. Examples of suitable heteroaryl include oxazole and oxadiazole, such as 1,2,4-oxadiazole and 1,3,4-oxadiazole. In certain embodiments, R³ is substituted with a single (C1-C4)-alkyl-, such as methyl or ethyl.

In some embodiments, the present invention provides a compound of formula I or I-A, wherein Y is —CH═; X and the two carbon atoms designated by α and β together form a phenyl ring substituted with 1 substituent selected from halogen (such as —Cl and —Br) and —OR where R is (C1-C4)-alkyl- (such as methyl); R² is (C1-C4)-alkyl- (such as methyl or ethyl); R³ is selected from the group consisting of (1) (C1-C4)-alkyl-, substituted with 1 or 2 halogens (such as —F), (2) —C(O)OR, wherein R is (C1-C4)-alkyl- (such as ethyl), (3) —C(O)N(R)₂, wherein each R is independently (C1-C4)-alkyl- (such as ethyl), or wherein the two R groups together with the nitrogen atom to which they are bound optionally form a 5-membered non-aromatic ring (such as a pyrrolidine ring), and (4) 5-membered heteroaryl-ring having two nitrogen atoms and one oxygen atom (such as 1,2,4-oxadiazole) where said 5-membered heteroaryl ring is substituted with one (C1-C4)-alkyl- (such as methyl). In some of the above embodiments for a compound of formula I or I-A, Y is —CH═; X and the two carbon atoms designated by α and β together form a phenyl ring substituted with —OMe, —Cl or —Br; R² is methyl or ethyl; R³ is selected from —CONEt₂ and —C(O)OEt. In some of the above embodiments for a compound of formula I or I-A, the compound is not:

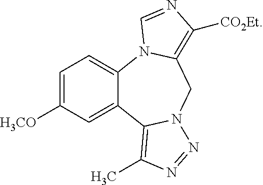 [SMILES: CCOC(=O)c1ncn2c1Cn1nnc(C)c1-c1cc(OC)ccc1-2]

Examples of particular compounds of the present invention include:

| Compound | Structure |
|---|---|
| 1 | 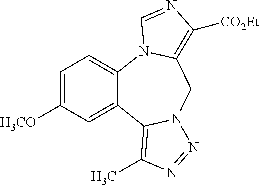 [SMILES: CCOC(=O)c1ncn2c1Cn1nnc(C)c1-c1cc(OC)ccc1-2] |
| 2 | 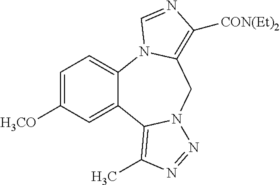 |
| 3 | 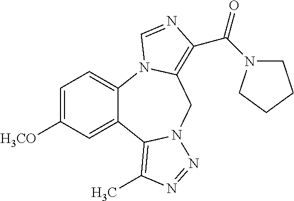 [SMILES: COc1ccc2c(c1)-c1c(C)nnn1Cc1c(C(=O)N3CCCC3)ncn1-2] |
| 4 | 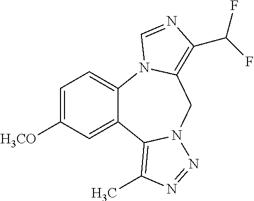 [SMILES: COc1ccc2c(c1)-c1c(C)nnn1Cc1c(C(F)F)ncn1-2] |
| 5 | 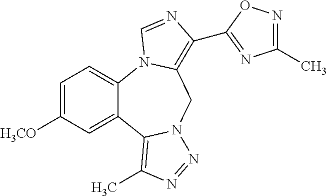 [SMILES: COc1ccc2c(c1)-c1c(C)nnn1Cc1c(-c3nc(C)no3)ncn1-2] |
| 6 | 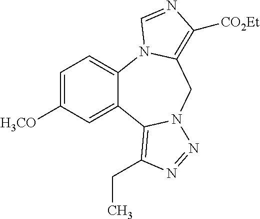 [SMILES: CCOC(=O)c1ncn2c1Cn1nnc(CC)c1-c1cc(OC)ccc1-2] |
| 7 | 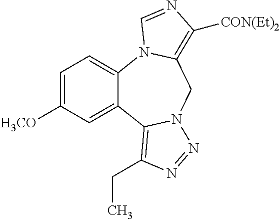 |
| 8 | 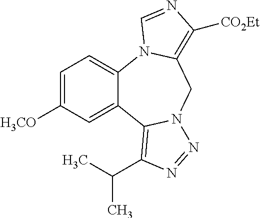 [SMILES: CCOC(=O)c1ncn2c1Cn1nnc(C(C)C)c1-c1cc(OC)ccc1-2] |
| 9 | 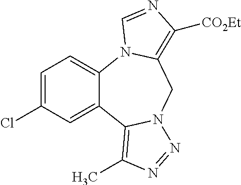 [SMILES: CCOC(=O)c1ncn2c1Cn1nnc(C)c1-c1cc(Cl)ccc1-2] |
| 10 | 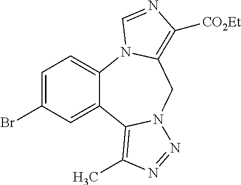 [SMILES: CCOC(=O)c1ncn2c1Cn1nnc(C)c1-c1cc(Br)ccc1-2] |
| 11 | 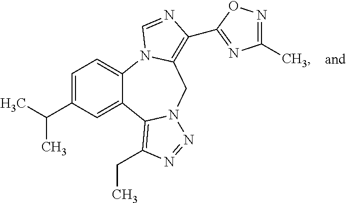 [SMILES: CCc1nnn2c1-c1cc(C(C)C)ccc1-n1cnc(-c3nc(C)no3)c1C2 ; "and"] |
| 12 | 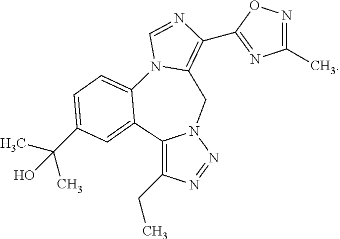 [SMILES: CCc1nnn2c1-c1cc(C(C)(C)O)ccc1-n1cnc(-c3nc(C)no3)c1C2] |

The invention also includes various combinations of R¹, R² and R³ as described above. These combinations can in turn be combined with any or all of the values of the other variables described above. For example, R¹ can be —OR or halogen and R² can be (C1-C4)-alkyl-, and optionally R³ is —C(O)OR, or —C(O)N(R)₂. In another example, R¹ is —OR or halogen and R² is (C1-C4)-alkyl-, and R³ is a 5- or 6-membered heteroaryl. For each of above examples, compounds can have the specific values of the groups described above.

Any embodiment given herein is also intended to represent unlabeled forms as well as isotopically labeled forms of the compounds, unless otherwise indicated. Isotopically labeled compounds have structures depicted by the formulas given herein except that one or more atoms are replaced by an atom having a selected atomic mass or mass number. Examples of isotopes that can be incorporated into compounds of the invention include isotopes of hydrogen, carbon, nitrogen, oxygen, phosphorous, fluorine, and chlorine, such as ²H, ³H, ¹¹C, ¹³C, ¹⁴C, ¹⁵N, ¹⁸F, ³¹P, ³²P, ³⁵S, ³⁶Cl, ¹²⁵I, respectively. The invention includes various isotopically labeled compounds as defined herein, for example those into which radioactive isotopes, such as ³H, ¹³C, and ¹⁴C, are present. Such isotopically labeled compounds are useful in metabolic studies (preferably with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F or labeled compound may be particularly preferred for PET or SPECT studies. Isotopically labeled compounds of this invention and prodrugs thereof can generally be prepared by carrying out the procedures disclosed in the schemes or in the examples and preparations described below by substituting a readily available isotopically labeled reagent for a non-isotopically labeled reagent.

Any of the individual embodiments recited above may define formula I, I-A, II or II-A individually or be combined to produce a preferred embodiment of this invention.

In another embodiment, the present invention provides a pharmaceutical composition comprising a pharmaceutically acceptable carrier and a compound of formula I, I-A, II or II-A or pharmaceutically acceptable salt form thereof.

Also, the basic nitrogen-containing groups may be quaternized with such agents as lower alkyl halides, such as methyl, ethyl, propyl, and butyl chloride, bromides and iodides; dialkyl sulfates, such as dimethyl, diethyl, dibutyl and diamyl sulfates, long chain halides such as decyl, lauryl, myristyl and stearyl chlorides, bromides and iodides, aralkyl halides, such as benzyl and phenethyl bromides and others. Water or oil-soluble or dispersible products are thereby obtained.

Pharmaceutically acceptable carriers that may be used in these compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

### (3) General Synthetic Methodology

The compounds of this invention may be prepared in general by methods known to those skilled in the art. Schemes 1-4 below provide general synthetic routes for the preparation of compounds of formula I or I-A. Other equivalent schemes, which will be readily apparent to the ordinary skilled organic chemist, may alternatively be used to synthesize various portions of the molecules as illustrated by the general schemes below.

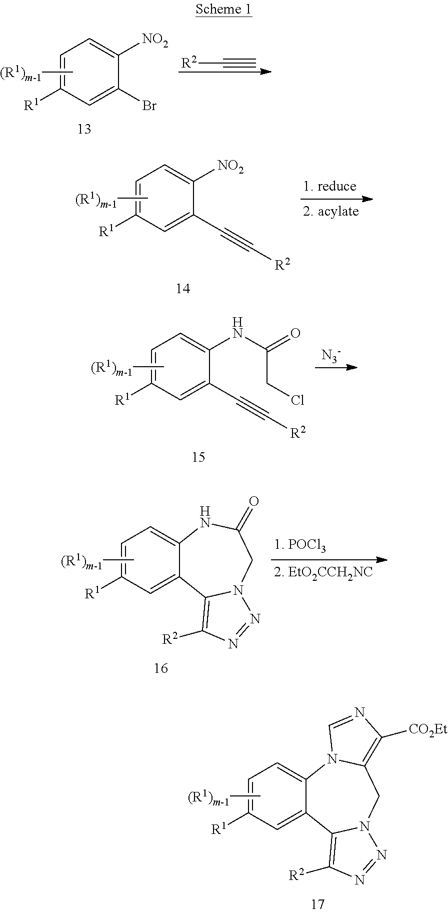

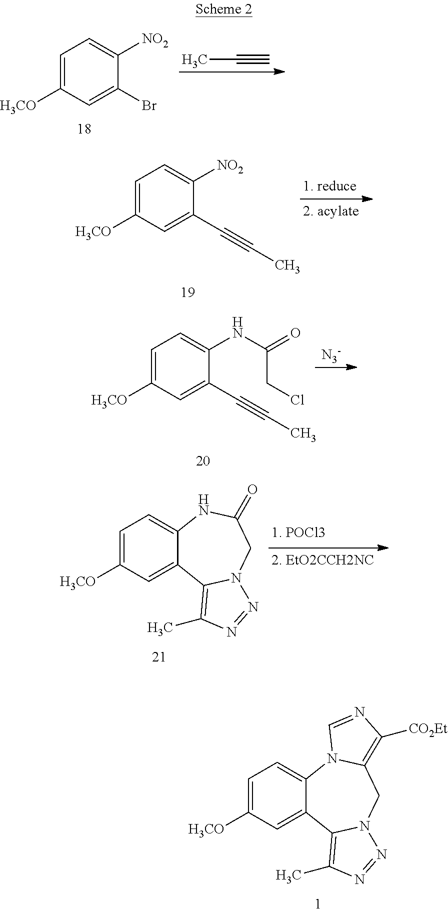

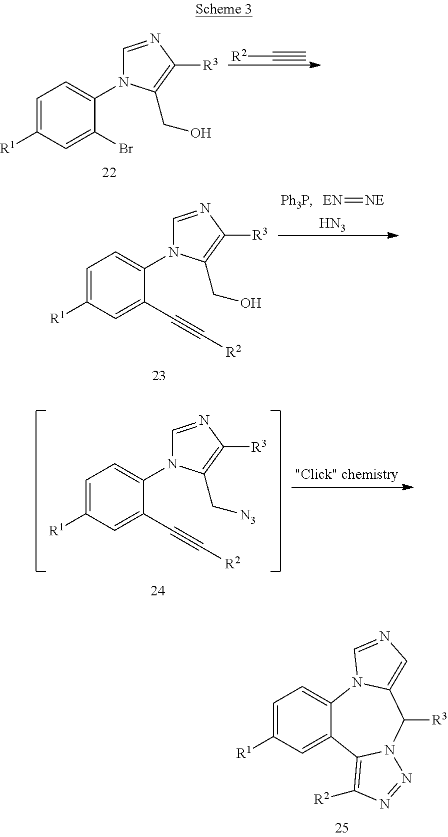

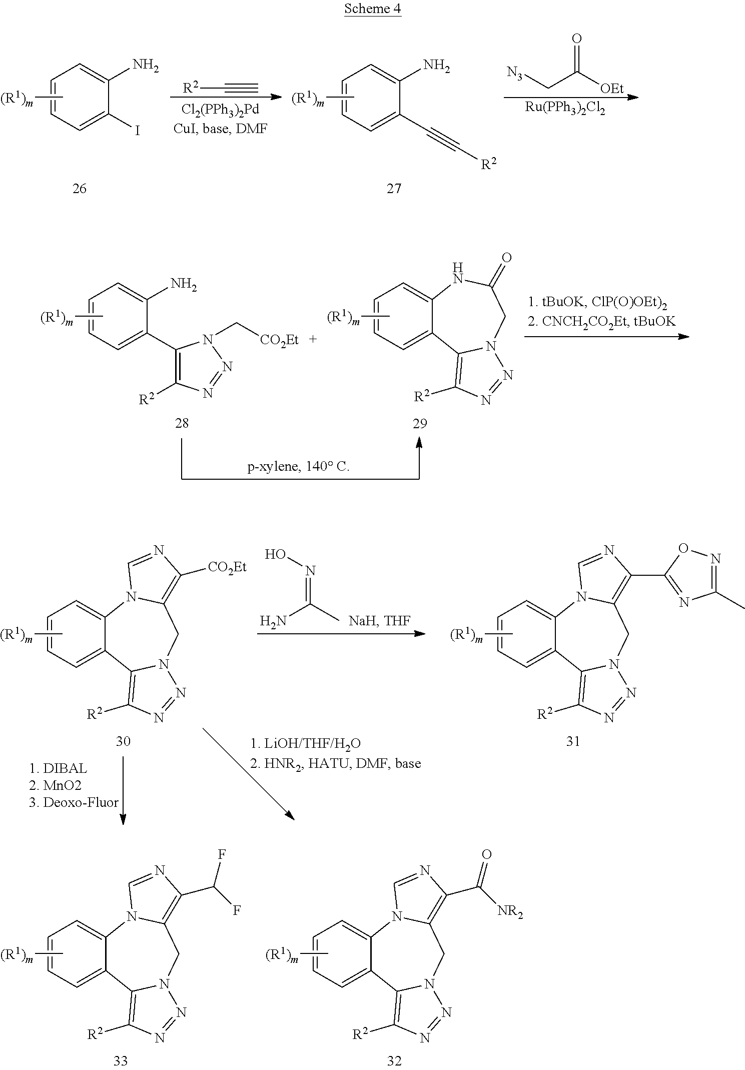

As would be recognized by skilled practitioners, compounds of formula I or I-A with Y, ring formed by X and the two carbon atoms designated by α and β, R¹, R² and R³ other than those depicted above may be prepared by varying chemical reagents or the synthetic routes.

### (4) Methods of Assessing Cognitive Impairment

Animal models serve as an important resource for developing and evaluating treatments for CNS disorders with cognitive impairment. Features that characterize cognitive impairment in animal models typically extend to cognitive impairment in humans. Efficacy in such animal models is, thus, expected to be predictive of efficacy in humans. The extent of cognitive impairment in an animal model for a CNS disorder, and the efficacy of a method of treatment for said CNS disorder may be tested and confirmed with the use of a variety of cognitive tests.

A Radial Arm Maze (RAM) behavioral task is one example of a cognitive test, specifically testing spacial memory (Chappell et al. Neuropharmacology 37: 481-487, 1998). The RAM apparatus consists of, e.g., eight equidistantly spaced arms. A maze arm projects from each facet of a center platform. A food well is located at the distal end of each arm. Food is used as a reward. Blocks can be positioned to prevent entry to any arm. Numerous extra maze cues surrounding the apparatus may also be provided. After habituation and training phases, spatial memory of the subjects may be tested in the RAM under control or test compound-treated conditions. As a part of the test, subjects are pretreated before trials with a vehicle control or one of a range of dosages of the test compound. At the beginning of each trial, a subset of the arms of the eight-arm maze is blocked. subjects are allowed to obtain food on the unblocked arms to which access is permitted during this initial “information phase” of the trial. subjects are then removed from the maze for a delay period, e.g., a 60 second delay, a 15 minute delay, a one-hour delay, a two-hour delay, a six hour delay, a 24 hour delay, or longer) between the information phase and the subsequent “retention test,” during which the barriers on the maze are removed, thus allowing access to all eight arms. After the delay period, subjects are placed back onto the center platform (with the barriers to the previously blocked arms removed) and allowed to obtain the remaining food rewards during this retention test phase of the trial. The identity and configuration of the blocked arms vary across trials. The number of “errors” the subjects make during the retention test phase is tracked. An error occurs in the trial if the subjects entered an arm from which food had already been retrieved in the pre-delay component of the trial, or if it re-visits an arm in the post-delay session that had already been visited. A fewer number of errors would indicate better spatial memory. The number of errors made by the test subject, under various test compound treatment regimes, can then be compared for efficacy of the test compound in treating a CNS disorder with cognitive impairment.

Another cognitive test that may be used to assess the effects of a test compound on the cognitive impairment of a CNS disorder model animal is the Morris water maze. A water maze is a pool surrounded with a novel set of patterns relative to the maze. The training protocol for the water maze may be based on a modified water maze task that has been shown to be hippocampal-dependent (de Hoz et al., Eur. J. Neurosci., 22:745-54, 2005; Steele and Morris, Hippocampus 9:118-36, 1999). The subject is trained to locate a submerged escape platform hidden underneath the surface of the pool. During the training trial, a subject is released in the maze (pool) from random starting positions around the perimeter of the pool. The starting position varies from trial to trial. If the subject does not locate the escape platform within a set time, the experimenter guides and places the subject on the platform to “teach” the location of the platform. After a delay period following the last training trial, a retention test in the absence of the escape platform is given to assess spatial memory. The subject's level of preference for the location of the (now absent) escape platform, as measured by, e.g., the time spent in that location or the number of crossings of that location made by the mouse, indicates better spatial memory, i.e., treatment of cognitive impairment. The preference for the location of the escape platform under different treatment conditions, can then be compared for efficacy of the test compound in treating a CNS disorder with cognitive impairment.

### (5) Age-Related Cognitive Impairment

This invention provides methods and compositions for treating age-related cognitive impairment or the risk thereof using a α5-containing GABA_{A} R agonist and analogs, derivatives, and pharmaceutically acceptable salts and solvates thereof. In certain embodiments, treatment comprises alleviation, amelioration or slowing the progression, of one or more symptoms associated with age-related cognitive impairment. In certain embodiments, treatment of age-related cognitive impairment comprises slowing the conversion of age-related cognitive impairment (including, but not limited to MCI, ARCD and AAMI) into dementia (e.g., AD). The methods and compositions may be used for human patients in clinical applications in the treating age-related cognitive impairment in conditions such as MCI, ARCD and AAMI or for the risk thereof. The dose of the composition and dosage interval for the method is, as described herein, one that is safe and efficacious in those applications.

In some embodiments, a subject to be treated by the methods and compositions of this invention exhibits age-related cognitive impairment or is at risk of such impairment. In some embodiments, the age-related cognitive impairment includes, without limitation, Age-Associated Memory Impairment (AAMI), Mild Cognitive Impairment (MCI) and Age-related Cognitive Decline (ARCD).

Animal models serve as an important resource for developing and evaluating treatments for such age-related cognitive impairments. Features that characterize age-related cognitive impairment in animal models typically extend to age-related cognitive impairment in humans. Efficacy in such animal models is, thus, expected to be predictive of efficacy in humans.

Various animal models of age-related cognitive impairment are known in the art. For example, extensive behavioral characterization has identified a naturally occurring form of cognitive impairment in an outbred strain of aged Long-Evans rats (Charles River Laboratories; Gallagher et al., Behav. Neurosci. 107:618-626, (1993)). In a behavioral assessment with the Morris Water Maze (MWM), rats learn and remember the location of an escape platform guided by a configuration of spatial cues surrounding the maze. The cognitive basis of performance is tested in probe trials using measures of the animal's spatial bias in searching for the location of the escape platform. Aged rats in the study population have no difficulty swimming to a visible platform, but an age-dependent impairment is detected when the platform is camouflaged, requiring the use of spatial information. Performance for individual aged rats in the outbred Long-Evans strain varies greatly. For example, a proportion of those rats perform on a par with young adults. However, approximately 40-50% fall outside the range of young performance. This variability among aged rats reflects reliable individual differences. Thus, within the aged population some animals are cognitively impaired and designated aged-impaired (AI) and other animals are not impaired and are designated aged-unimpaired (AU). See, e.g., Colombo et al., Proc. Natl. Acad. Sci. 94: 14195-14199, (1997); Gallagher and Burwell, Neurobiol. Aging 10: 691-708, (1989); Gallagher et al. Behav. Neurosci. 107:618-626, (1993); Rapp and Gallagher, Proc. Natl. Acad. Sci. 93: 9926-9930, (1996); Nicolle et al., Neuroscience 74: 741-756, (1996); Nicolle et al., J. Neurosci. 19: 9604-9610, (1999); International Patent Publication WO2007/019312 and International Patent Publication WO 2004/048551. Such an animal model of age-related cognitive impairment may be used to assay the effectiveness of the methods and compositions this invention in treating age-related cognitive impairment.

The efficacy of the methods and compositions of this invention in treating age-related cognitive impairment may be assessed using a variety of cognitive tests, including the Morris water maze and the radial arm maze, as discussed above.

### (6) Dementia

This invention also provides methods and compositions for treating dementia using a α5-containing GABA_{A} R agonist and analogs, derivatives, and pharmaceutically acceptable salts and solvates thereof. In certain embodiments, treatment comprises alleviation, amelioration, or slowing the progression of one or more symptoms associated with dementia. In certain embodiments, the symptom to be treated is cognitive impairment. In certain embodiments, the dementia is Alzheimer's disease (AD), vascular dementia, dementia with Lewy bodies, or frontotemporal dementia. The methods and compositions may be used for human patients in clinical applications in treating dementia. The dose of the composition and dosage interval for the method is, as described herein, one that is safe and efficacious in those applications.

Animal models serve as an important resource for developing and evaluating treatments for dementia. Features that characterize dementia in animal models typically extend to dementia in humans. Thus, efficacy in such animal models is expected to be predictive of efficacy in humans. Various animal models of dementia are known in the art, such as the PDAPP, Tg2576, APP23, TgCRND8, J20, hPS2 Tg, and APP+PS1 transgenic mice. Sankaranarayanan, Curr. Top. Medicinal Chem. 6: 609-627, 2006; Kobayashi et al. Genes Brain Behav. 4: 173-196. 2005; Ashe and Zahns, Neuron. 66: 631-45, 2010. Such animal models of dementia may be used to assay the effectiveness of the methods and compositions of this invention of the invention in treating dementia.

The efficacy of the methods and compositions of this invention in treating dementia, or cognitive impairment associated with dementia, may be assessed in animals models of dementia using a variety of cognitive tests known in the art, including the Morris water maze and the radial arm maze, as discussed above.

### (7) Post Traumatic Stress Disorder

This invention also provides methods and compositions for treating post traumatic stress disorder (PTSD) using a α5-containing GABA_{A} R agonist and analogs, derivatives, and pharmaceutically acceptable salts and solvates thereof. In certain embodiments, treatment comprises alleviation, amelioration, or slowing the progression of one or more symptoms associated with PTSD. In certain embodiments, the symptom to be treated is cognitive impairment. The methods and compositions may be used for human patients in clinical applications in treating PTSD. The dose of the composition and dosage interval for the method is, as described herein, one that is safe and efficacious in those applications.

Patients with PTSD (and, to a lesser degree trauma-exposed patients without PTSD) have smaller hippocampal volumes (Woon et al., Prog. Neuro-Psychopharm. & Biological Psych. 34, 1181-1188; Wang et al., Arch. Gen. Psychiatry 67:296-303, 2010). PTSD is also associated with impaired cognitive performance. Older individuals with PTSD have greater declines in cognitive performance relative to control patients (Yehuda et al., Bio. Psych. 60: 714-721, 2006) and have a greater likelihood of developing dementia (Yaffe et al., Arch. Gen. Psych. 678: 608-613, 2010).

Animal models serve as an important resource for developing and evaluating treatments for PTSD. Features that characterize PTSD in animal models typically extend to PTSD in humans. Thus, efficacy in such animal models is expected to be predictive of efficacy in humans. Various animal models of PTSD are known in the art.

One rat model of PTSD is Time-dependent sensitization (TDS). TDS involves exposure of the animal to a severely stressful event followed by a situational reminder of the prior stress. The following is an example of TDS. Rats are placed in a restrainer, then placed in a swim tank and made to swim for a period of time, e.g., 20 min. Following this, each rat is then immediately exposed to a gaseous anesthetic until loss of consciousness, and finally dried. The animals are left undisturbed for a number of days, e.g., one week. The rats are then exposed to a “restress” session consisting of an initial stressor, e.g., a swimming session in the swim tank (Liberzon et al., Psychoneuroendocrinology 22: 443-453, 1997; Harvery et al., Psychopharmacology 175:494-502, 2004). TDS results in an enhancement of the acoustic startle response (ASR) in the rat, which is comparable to the exaggerated acoustic startle that is a prominent symptom of PTSD (Khan and Liberzon, Psychopharmacology 172: 225-229, 2004). Such animal models of PTSD may be used to assay the effectiveness of the methods and compositions of this invention of the invention in treating PTSD.

The efficacy of the methods and compositions of this invention in treating PTSD, or cognitive impairment associated with PTSD, may also be assessed in animals models of PTSD using a variety of other cognitive tests known in the art, including the Morris water maze and the radial arm maze, as discussed above.

### (8) Schizophrenia

This invention additionally provides methods and compositions for treating schizophrenia using a α5-containing GABA_{A} R agonist and analogs, derivatives, and pharmaceutically acceptable salts and solvates thereof. In certain embodiments, treatment comprises alleviation, amelioration or slowing the progression, of one or more symptoms associated with schizophrenia. In certain embodiments, the symptom to be treated is cognitive impairment. The methods and compositions may be used for human patients in clinical applications in treating schizophrenia. The dose of the composition and dosage interval for the method is, as described herein, one that is safe and efficacious in those applications.

Animal and human studies demonstrate that GABA signaling is reduced in schizophrenia, for examples in various areas of the cerebral cortex and hippocampus. See, e.g., Akbarian et al., Arch. Gen. Psychiatry 52:258-266, 1995; Volk et al., Arch. Gen. Psychiatry 57:237-245, 2000; Hashimoto et al., J. Neurosci. 23:6315-6326, 2003; Hashimoto et al., Mol. Psychiatry. 13:147-161.2008; Lodge et al., J. Neurosci., 29:2344-2354, 2009; Yoon et al., J. Neurosci. 30: 3777-81, 2010. Cognitive impairments are also associated with schizophrenia. They precede the onset of psychosis and are present in non-affected relatives. The cognitive impairments associated with schizophrenia constitute a good predictor for functional outcome and are a core feature of the disorder. Cognitive features in schizophrenia reflect dysfunction in frontal cortical and hippocampal circuits. Patients with schizophrenia also present hippocampal pathologies such as reductions in hippocampal volume, reductions in neuronal size and dysfunctional hyperactivity. An imbalance in excitation and inhibition in these brain regions has also been documented in schizophrenic patients suggesting that drugs targeting inhibitory mechanisms could be therapeutic. See, e.g., Guidotti et al., Psychopharmacology 180: 191-205, 2005; Zierhut, Psych. Res. Neuroimag. 183:187-194, 2010; Wood et al., NeuroImage 52:62-63, 2010; Vinkers et al., Expert Opin. Investig. Drugs 19:1217-1233, 2009; Young et al., Pharmacol. Ther. 122:150-202, 2009. In particular, compounds that selectively and positively modulate the action of GABA_{A} receptors comprising α5 subunits have been proposed as therapeutic agents that will contribute to the anxiolytic, antipanic and anticonvulsant actions without producing sedation, amnesia, or tolerance (Guidotti et al., Psychopharmacology 180: 191-205, 2005).

Animal models serve as an important resource for developing and evaluating treatments for schizophrenia. Features that characterize schizophrenia in animal models typically extend to schizophrenia in humans. Thus, efficacy in such animal models is expected to be predictive of efficacy in humans. Various animal models of schizophrenia are known in the art.

One animal model of schizophrenia is protracted treatment with methionine. Methionine-treated mice exhibit deficient expression of GAD67 in frontal cortex and hippocampus, similar to those reported in the brain of postmortem schizophrenia patients. They also exhibit prepulse inhibition of startle and social interaction deficits (Tremonlizzo et al., PNAS, 99: 17095-17100, 2002). Another animal model of schizophrenia is methylaoxymethanol acetate (MAM)-treatment in rats. Pregnant female rats are administered MAM (20 mg/kg, intraperitoneal) on gestational day 17. MAM-treatment recapitulate a pathodevelopmental process to schizophrenia-like phenotypes in the offspring, including anatomical changes, behavioral deficits and altered neuronal information processing. More specifically, MAM-treated rats display a decreased density of parvalbumin-positive GABAergic interneurons in portions of the prefrontal cortex and hippocampus. In behavioral tests, MAM-treated rats display reduced latent inhibition. Latent inhibition is a behavioral phenomenon where there is reduced learning about a stimulus to which there has been prior exposure with any consequence. This tendency to disregard previously benign stimuli, and reduce the formation of association with such stimuli is believed to prevent sensory overload. Low latent inhibition is indicative of psychosis. Latent inhibition may be tested in rats in the following manner. Rats are divided into two groups. One group is pre-exposed to a tone over multiple trials. The other group has no tone presentation. Both groups are then exposed to an auditory fear conditioning procedure, in which the same tone is presented concurrently with a noxious stimulus, e.g. an electric shock to the foot. Subsequently, both groups are presented with the tone, and the rats' change in locomotor activity during tone presentation is monitored. After the fear conditioning the rats respond to the tone presentation by strongly reducing locomotor activity. However, the group that has been exposed to the tone before the conditioning period displays robust latent inhibition: the suppression of locomotor activity in response to tone presentation is reduced. MAM-treated rats, by contrast show impaired latent inhibition. That is, exposure to the tone previous to the fear conditioning procedure has no significant effect in suppressing the fear conditioning. (see Lodge et al., J. Neurosci., 29:2344-2354, 2009) Such animal models of schizophrenia may be used to assay the effectiveness of the methods and compositions of the invention in treating schizophrenia.

The efficacy of the methods and compositions of this invention in treating schizophrenia, or cognitive impairment associated with schizophrenia, may also be assessed in animal models of schizophrenia using a variety of other cognitive tests known in the art, including the Morris water maze and the radial arm maze, as discussed above.

### (9) Cancer Therapy-Related Cognitive Impairment

This invention additionally provides methods and compositions for treating cancer therapy-related cognitive impairment using a α5-containing GABA_{A} R agonist and analogs, derivatives, and pharmaceutically acceptable salts and solvates thereof. In certain embodiments, treatment comprises alleviation, amelioration or slowing the progression, of one or more symptoms associated with cancer therapy-related cognitive impairment. The methods and compositions may be used for human patients in clinical applications in treating cancer therapy-related cognitive impairment. The dose of the composition and dosage interval for the method is, as described herein, one that is safe and efficacious in those applications.

Therapies that are used in cancer treatment, including chemotherapy, radiation, or combinations thereof, can cause cognitive impairment in patients, in such functions as memory, learning, and attention. Cytotoxicity and other adverse side-effects on the brain of cancer therapies are the basis for this form of cognitive impairment, which can persist for decades. (Dietrich et al., Oncologist 13:1285-95, 2008; Soussain et al., Lancet 374:1639-51, 2009).

Cognitive impairment following cancer therapies reflects dysfunction in frontal cortical and hippocampal circuits that are essential for normal cognition. In animal models, exposure to either chemotherapy or radiation adversely affects performance on tests of cognition specifically dependent on these brain systems, especially the hippocampus (Kim et al., J. Radiat. Res. 49:517-526, 2008; Yang et al., Neurobiol. Learning and Mem. 93:487-494, 2010). Thus, drugs targeting these cortical and hippocampal systems could be neuroprotective in patients receiving cancer therapies and efficacious in treating symptoms of cognitive impairment that may last beyond the interventions used as cancer therapies.

Animal models serve as an important resource for developing and evaluating treatments for cancer therapy-related cognitive impairment. Features that characterize cancer therapy-related cognitive impairment in animal models typically extend to cancer therapy-related cognitive impairment in humans. Thus, efficacy in such animal models is expected to be predictive of efficacy in humans. Various animal models of cancer therapy-related cognitive impairment are known in the art.

Examples of animal models of cancer therapy-related cognitive impairment include treating animals with anti-neoplastic agents such as cyclophosphamide (CYP) or with radiation, e.g., ⁶⁰ Co gamma-rays. (Kim et al., J. Radiat. Res. 49:517-526, 2008; Yang et al., Neurobiol. Learning and Mem. 93:487-494, 2010). The cognitive function of animal models of cancer therapy-related cognitive impairment may then be tested with cognitive tests to assay the effectiveness of the methods and compositions of the invention in treating cancer therapy-related cognitive impairment. The efficacy of the methods and compositions of this invention in treating cancer therapy-related cognitive impairment may be assessed using a variety of cognitive tests known in the art, including the Morris water maze and the radial arm maze, as discussed above.

### (10) Research Domain Criteria (RDoC)

This invention further provides methods and compositions for treating impairment in neurological disorders and neuropsychiatric conditions using a α5-containing GABA_{A} R agonists and analogs, derivatives, and pharmaceutically acceptable salts and solvates thereof. In certain embodiments, treatment comprises alleviation, amelioration or slowing the progression, of one or more symptoms associated with such impairment.

Research Domain Criteria (RDoC) are expected to augment clinical criteria, such as DSM and ICD, for diagnosis of disease and disorders affecting the nervous system (see, e.g., Am. J. Psychiatry 167:7 (2010)). The RDoC is intended to provide classification based on discoveries in genomics and neuroscience as well as clinical observation. The high expression of α5-containing GABA_{A} receptors in specific neural circuits in the nervous system could be therapeutic targets for neural circuit dysfunction identified under RDoC.

### (11) Assays for GABA_{A} α5 Subunit Binding and Receptor Agonist Activity

The affinity of test compounds for a GABA_{A} receptor comprising the GABA_{A} α5 subunit may be determined using receptor binding assays that are known in the art. See, e.g., U.S. Pat. No. 7,642,267 and U.S. Pat. No. 6,743,789, which are incorporated herein by reference.

The activity of the test compounds as a α5-containing GABA_{A} R agonist may be tested by electrophysiological methods known in the art. See, e.g., U.S. Pat. No. 7,642,267 and Guidotti et al., Psychopharmacology 180: 191-205, 2005. Agonist activity may be tested, for examples, by assaying GABA-induced chloride ion conductance of GABA_{A} receptors comprising the GABA_{A} α5 subunit. Cells expressing such receptors may be exposed to an effective amount of a compound of the invention. Such cells may be contacted in vivo with compounds of the invention through contact with a body fluid containing the compound, for example through contact with cerebrospinal fluid. In vitro tests may be done by contacting cells with a compound of the invention in the presence of GABA. Increased GABA-induced chloride conductance in cells expressing GABA_{A} receptors comprising the GABA_{A} α5 subunit in the presence of the test compound would indicate agonist activity of said compound. Such changes in conductance may be detected by, e.g., using a voltage-clamp assay performed on Xenopus oocytes injected with GABA_{A} receptor subunit mRNA (including GABA_{A} α5 subunit RNA), HEK 293 cells transfected with plasmids encoding GABA_{A} receptor subunits, or in vivo, ex vivo, or cultured neurons.

### (12) Compositions and Modes of Administration

It will be appreciated that compounds and agents used in the compositions and methods of the present invention preferably should readily penetrate the blood-brain barrier when peripherally administered. Compounds which cannot penetrate the blood-brain barrier, however, can still be effectively administered directly into the central nervous system, e.g., by an intraventricular route.

In some embodiments of this invention, the α5-containing GABA_{A} R agonist is formulated with a pharmaceutically acceptable carrier. In other embodiments, no carrier is used. For example, the α5-containing GABA_{A} R agonist can be administered alone or as a component of a pharmaceutical formulation (therapeutic composition). The α5-containing GABA_{A} R agonist may be formulated for administration in any convenient way for use in human medicine.

In some embodiments, the therapeutic methods of the invention include administering the composition of a compound or agent topically, systemically, or locally. For example, therapeutic compositions of compounds or agents of the invention may be formulated for administration by, for example, injection (e.g., intravenously, subcutaneously, or intramuscularly), inhalation or insufflation (either through the mouth or the nose) or oral, buccal, sublingual, transdermal, nasal, or parenteral administration. The compositions of compounds or agents described herein may be formulated as part of an implant or device, or formulated for slow or extended release. When administered parenterally, the therapeutic composition of compounds or agents for use in this invention is preferably in a pyrogen-free, physiologically acceptable form. Techniques and formulations generally may be found in Remington's Pharmaceutical Sciences, Meade Publishing Co., Easton, Pa.

In certain embodiments, pharmaceutical compositions suitable for parenteral administration may comprise the α5-containing GABA_{A} R agonist in combination with one or more pharmaceutically acceptable sterile isotonic aqueous or non-aqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents. Examples of suitable aqueous and non-aqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

A composition comprising a α5-containing GABA_{A} R agonist may also contain adjuvants, such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption, such as aluminum monostearate and gelatin.

In certain embodiments of the invention, compositions comprising a α5-containing GABA_{A} R agonist can be administered orally, e.g., in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and the like, each containing a predetermined amount of the α5-containing GABA_{A} R agonist as an active ingredient.

In solid dosage forms for oral administration (capsules, tablets, pills, dragees, powders, granules, and the like), one or more compositions comprising the α5-containing GABA_{A} R agonist may be mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose, and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

Liquid dosage forms for oral administration include pharmaceutically acceptable emulsions, microemulsions, solutions, suspensions, syrups, and elixirs. In addition to the α5-containing GABA_{A} R agonist, the liquid dosage forms may contain inert diluents commonly used in the art, such as water or other solvents, solubilizing agents and emulsifiers, such as ethyl alcohol (ethanol), isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, coloring, perfuming, and preservative agents.

Suspensions, in addition to the active compounds, may contain suspending agents such as ethoxylated isostearyl alcohols, polyoxyethylene sorbitol, and sorbitan esters, microcrystalline cellulose, aluminum metahydroxide, bentonite, agar-agar and tragacanth, and mixtures thereof.

As described above, the compounds, agents, and compositions thereof may be administered for slow, controlled or extended release. The term “extended release” is widely recognized in the art of pharmaceutical sciences and is used herein to refer to a controlled release of an active compound or agent from a dosage form to an environment over (throughout or during) an extended period of time, e.g. greater than or equal to one hour. An extended release dosage form will release drug at substantially constant rate over an extended period of time or a substantially constant amount of drug will be released incrementally over an extended period of time. The term “extended release” used herein includes the terms “controlled release,” “prolonged release,” “sustained release,” or “slow release,” as these terms are used in the pharmaceutical sciences. In some embodiments, the extended release dosage is administered in the form of a patch or a pump.

A person of ordinary skill in the art, such as a physician, is readily able to determine the required amount of α5-containing GABA_{A} R agonist (s) to treat the subject using the compositions and methods of this invention. It is understood that the dosage regimen will be determined for an individual, taking into consideration, for example, various factors that modify the action of α5-containing GABA_{A} R agonist, the severity or stage of the disease, route of administration, and characteristics unique to the individual, such as age, weight, size, and extent of cognitive impairment.

It is well-known in the art that normalization to body surface area is an appropriate method for extrapolating doses between species. To calculate the human equivalent dose (HED) from a dosage used in the treatment of age-dependent cognitive impairment in rats, the formula HED (mg/kg)=rat dose (mg/kg)×0.16 may be employed (see Estimating the Safe Starting Dose in Clinical Trials for Therapeutics in Adult Healthy Volunteers, December 2002, Center for Biologics Evaluation and Research). For example, using that formula, a dosage of 10 mg/kg in rats is equivalent to 1.6 mg/kg in humans. This conversion is based on a more general formula HED=animal dose in mg/kg×(animal weight in kg/human weight in kg) 0.33

In certain embodiments of the invention, the dose of the α5-containing GABA_{A} R agonist is between 0.0001 and 100 mg/kg/day (which, given a typical human subject of 70 kg, is between 0.007 and 7000 mg/day).

In certain embodiments of the invention, the interval of administration is once every 12 or 24 hours. Administration at less frequent intervals, such as once every 6 hours, may also be used.

If administered by an implant, a device or a slow or extended release formulation, the α5-containing GABA_{A} R agonist can be administered one time, or one or more times periodically throughout the lifetime of the patient as necessary. Other administration intervals intermediate to or shorter than these dosage intervals for clinical applications may also be used and may be determined by one skilled in the art following the methods of this invention.

Desired time of administration can be determined by routine experimentation by one skilled in the art. For example, the α5-containing GABA_{A} R agonist may be administered for a period of 1-4 weeks, 1-3 months, 3-6 months, 6-12 months, 1-2 years, or more, up to the lifetime of the patient.

In addition to α5-containing GABA_{A} R agonist, the compositions and methods of this invention can also include other therapeutically useful agents. These other therapeutically useful agents may be administered in a single formulation, simultaneously or sequentially with the α5-containing GABA_{A} R agonist according to the methods of the invention.

It will be understood by one of ordinary skill in the art that the compositions and methods described herein may be adapted and modified as is appropriate for the application being addressed and that the compositions and methods described herein may be employed in other suitable applications, and that such other additions and modifications will not depart from the scope hereof.

This invention will be better understood from the Examples which follow. However, one skilled in the art will readily appreciate that the specific methods and results discussed are merely illustrative of the invention as described more fully in the embodiments which follow thereafter.

### EXAMPLES

### Example 1

### Synthesis of Compound 1

### Step 1:

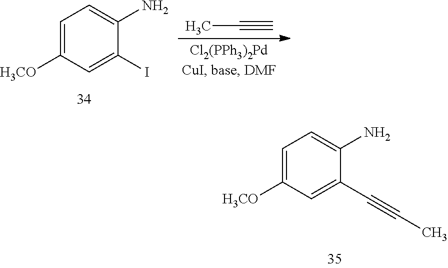

A thick-walled reaction vial was degassed and placed under a N₂ atmosphere. To it was added 2-iodo-4-methoxyphenylamine 34 (2.10 g, 8.43 nmol), CuI (0.161 g, 0.843 mmol), and bis-triphenylphosphine-palladium(11) chloride (0.292 g, 0.42 mmol). The flask was purged again with N₂ and cooled to −78° C. Methyl acetylene, as a gas (1.69 g, 42.2 mmol) was delivered into it. To the flask was slowly added THF (25 mL) via syringe. The reaction was warmed to RT and stirred for 16 h. The mixture was diluted with diethyl ether, washed with water, brine, and dried over MgSO₄. Filtration and concentration afforded 0.735 g (54%) of product 35 as a brown oil which was of sufficient purity as indicated by LC/MS and ¹H NMR to carry forth to the next step. (MS: [M+1=162]).

### Step 2:

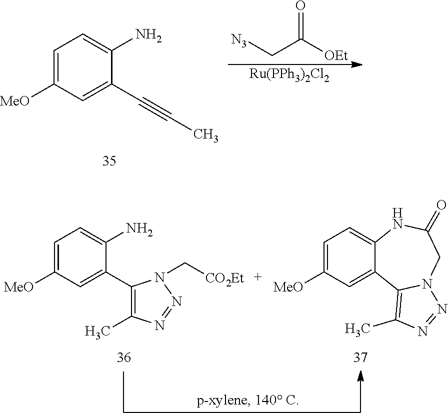 [SMILES: CC#Cc1cc(OC)ccc1N ; CCOC(=O)CN=[N+]=[N-] ; CCOC(=O)Cn1nnc(C)c1-c1cc(OC)ccc1N ; COc1ccc2c(c1)-c1c(C)nnn1CC(=O)N2 ; "Ru(PPh3)2Cl2"]

Compound 35 (1.83 g, 11.35 mmol) was placed under N₂ atmosphere and dissolved in DMF (10 mL). To it was added chlorocyclopentadienylbis-(triphenylphosphine)ruthenium(II) (0.405 g, 0.51 mmol) and a dropwise solution of ethyl azidoacetate (45 mL, 0.5M solution, 22.5 mmol). The reaction was purged with N₂ and stirred for 3 days at RT. As indicated by LC/MS, the reaction mixture consisted of starting material 35, triazole Click-adduct 36, and cyclized product 37 in a ratio of 11:26:13.

Additional quantities of chlorocyclopentadienylbis(triphenylphosphine)-ruthenium(II) (0.09 g) and ethyl azidoacetate (23 mL) were added and the reaction stirred for an additional 5 h. The mixture was diluted with diethyl ether and the organic phase was washed with saturated NaHCO₃. The aqueous phase was back-extracted with diethyl ether. The combined organic phase was dried over MgSO₄. The concentrated residue was purified by ISCO (gradient 100% hexanes to 100% EtOAc) to remove the starting material and afford a mixture of 36 and desired product 37 as a brown solid. To affect further cyclization of 36 to 37, the residue was dissolved in p-xylene (20 mL) and stirred at 140° C. for 15 h. The reaction was cooled to 0° C. and filtered to give 1.58 g compound 37 as a grey-white powder (57%) of sufficient purity to take on to the next step, (MS: [M+1=245]).

### Step 3:

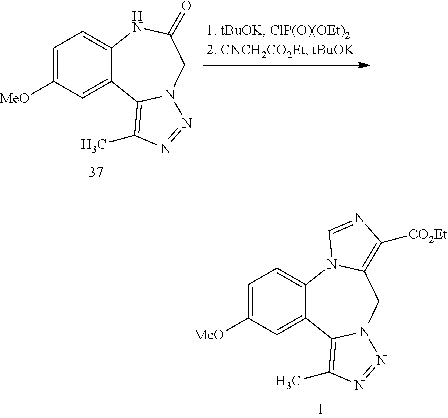 [SMILES: COc1ccc2c(c1)-c1c(C)nnn1CC(=O)N2 ; CCOC(=O)c1ncn2c1Cn1nnc(C)c1-c1cc(OC)ccc1-2]

Compound 37 (0.1285 g, 0.526 mmol) was dissolved in THF (2.5 mL) at −20° C. under a blanket of N₂. To it was added t-BuOK (97%, 76.7 mg, 0.684 mmol). Following dropwise addition of diethyl chlorophosphate (98.8 uL, 0.684 mmol), the mixture was stirred for 5 h while warming from −20 to 10° C. The reaction mixture was re-cooled to −78° C. and to it was added ethyl isocyanoacetate (80.5 uL, 0.736 mmol) followed by t-BuOK (97%, 76.7 mg, 0.684 mmol). The reaction was warmed from −78° C. to RT and then stirred overnight, quenched by addition of saturated NaHCO₃, and extracted with EtOAc. The organic phase was washed with saturated NaHCO₃, brine, then dried (MgSO₄) and concentrated to give a residue. Purification by ISCO (gradient EtOAc to 2% MeOH in EtOAc) afforded 56.9 mg (32%) of compound 1 as a yellow solid, (MS: [M+1=340]).

### Example 2

### Synthesis of Compound 2

### Step 1:

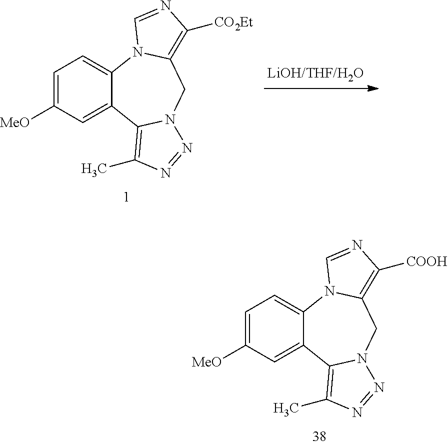 [SMILES: CCOC(=O)c1ncn2c1Cn1nnc(C)c1-c1cc(OC)ccc1-2 ; COc1ccc2c(c1)-c1c(C)nnn1Cc1c(C(=O)O)ncn1-2]

Compound 1 (221.8 mg, 0.654 mmol) and LiOH (31.3 mg, 1.31 mmol) were dissolved in 4:1 THF/H₂O (6.5 mL) and the mixture was stirred for 14 h at RT, concentrated, and neutralized with 0.4 N HCl and saturated NaHCO₃. The solution was stored overnight at 5° C. and the precipitate was collected by filtration to give the carboxylic acid 38 as a light yellow powder (179.5 mg, 88%) which was taken on directly in the next step, (MS: [M+1=312]).

### Step 2:

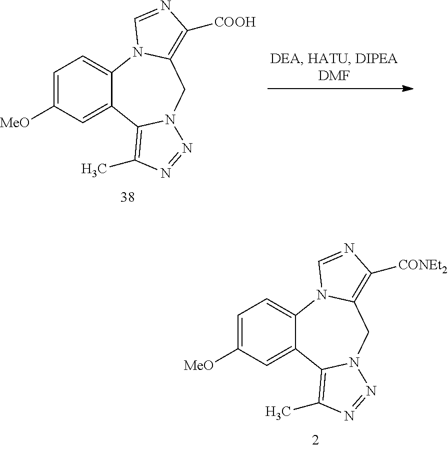

Carboxylic acid 38 (91.5 mg, 0.294 mmol) was dissolved in DMF (2 mL). To the solution was added diisopropylethylamine (0.154 mL, 0.882 mmol), O-(7-azabenzotriazol-1-yl)-N,N,N′,N′-tetramethyluronium hexafluorophosphate (167.6 mg, 0.441 mmol), and diethylamine (91.2 uL, 0.882 mmol) and the mixture was stirred at RT for 16 h. The mixture was diluted with EtOAc, washed with saturated NaHCO₃ and brine, and dried over MgSO₄. Filtration and evaporation of the filtrate gave crude product which was purified by ISCO (gradient hexanes/EtOAc) to give compound 2 as light brown amorphous solid (107.2 mg, 100%), (MS: [M+1=367]).

### Example 3

### Synthesis of Compound 3

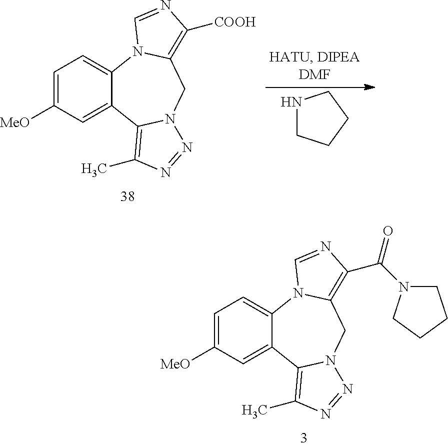 [SMILES: COc1ccc2c(c1)-c1c(C)nnn1Cc1c(C(=O)O)ncn1-2 ; C1CCNC1 ; COc1ccc2c(c1)-c1c(C)nnn1Cc1c(C(=O)N3CCCC3)ncn1-2]

Using a procedure that is substantially similar to the one as described in Example 2, Step 2, 86.1 mg of compound 38 was converted to compound 3: 100.7 mg, (100%), (MS: [M+1=365]).

### Example 4

### Synthesis of Compound 4

### Step 1:

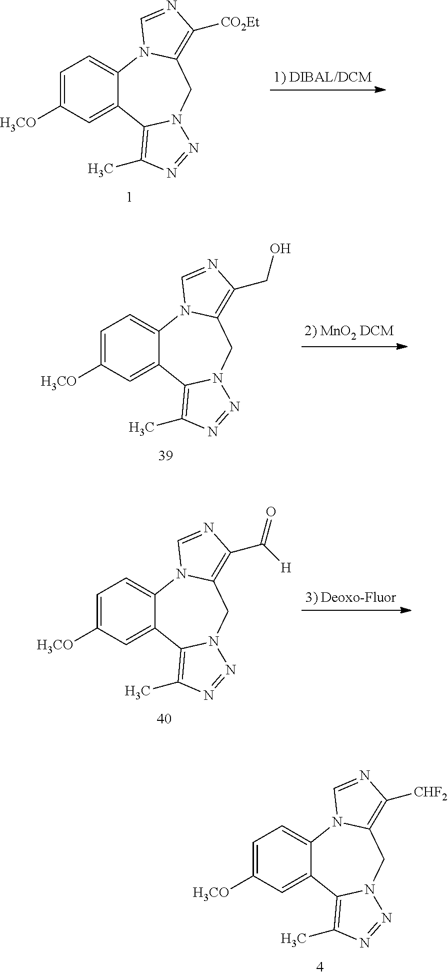 [SMILES: CCOC(=O)c1ncn2c1Cn1nnc(C)c1-c1cc(OC)ccc1-2 ; COc1ccc2c(c1)-c1c(C)nnn1Cc1c(CO)ncn1-2 ; COc1ccc2c(c1)-c1c(C)nnn1Cc1c(C=O)ncn1-2 ; COc1ccc2c(c1)-c1c(C)nnn1Cc1c(C(F)F)ncn1-2]

Compound 1 (0.169 g, 0.498 mmol) was dissolved in dichloromethane (2.5 mL) at 0° C. and treated with diisobutylaluminum hydride (1.49 mL, 1 M in THF, 1.49 mmol). The reaction was stirred for 3 h while warming to RT, quenched with methanol, concentrated, and the resulting residue was purified by ISCO (gradient DCM to 10% MeOH/DCM). The corresponding alcohol 39 (0.119 g, 80%) was formed as an off-white solid, (MS: [M+1=298]).

### Step 2:

Alcohol 39 (0.119 g, 0.401 mmol) was dissolved in dichloromethane (8 mL) and treated with MnO₂ (0.386 g, 4 mmol). The reaction was stirred at RT for 16 h, filtered, and concentrated to give 86.3 mg (73%) of the corresponding aldehyde 40 as a brownish solid which was of sufficient purity to take on to the next step, (MS: [M+1=296]).

### Step 3:

Aldehyde 40 (0.863 g, 0.292 mmol) was dissolved in dichloromethane (2 mL) at 0° C., treated with [bis(2-methoxyethyl)amino]-sulfur trifluoride (Deoxo-fluor) (0.646 g, 2.92 mmol), and stirred for 16 h at RT. The mixture was diluted with additional dichloromethane, washed with saturated NaHCO₃ and brine, the organic phase was dried over MgSO₄. Concentration afforded crude product which was purified by prep TLC (dichloromethane/MeOH 5%) to give compound 4 (55.7 mg, 60%) as an off-white solid, (MS: [M+1=318]).

### Example 5

### Synthesis of Compound 5

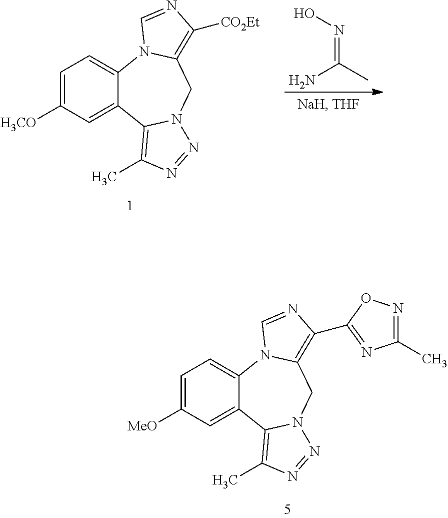

N-Hydroxyacetamidine (73.6 mg, 0.944 mmol) was dried by three consecutive dilutions and concentrations in toluene (3 mL each time). After drying in vacuo, to it was added THF (3 mL) and NaH (34.8 mg, 0.871 mmol). After stirring for 15 min at RT, compound 1 (246.4 mg, 0.726 mmol) was added and the resultant mixture was stirred for 2 h at RT then at 70° C. for 5 h. The mixture was cooled, diluted with ice water (20 mL) and allowed to sit overnight in the refrigerator. An off-white precipitate was vacuum filtered, washed with water, and dried in vacuo to give 120.5 mg (48%) of compound 5 as an off white solid. (MS: [M+1=350]).

### Example 6

### Synthesis of Compound 6

### Step 1:

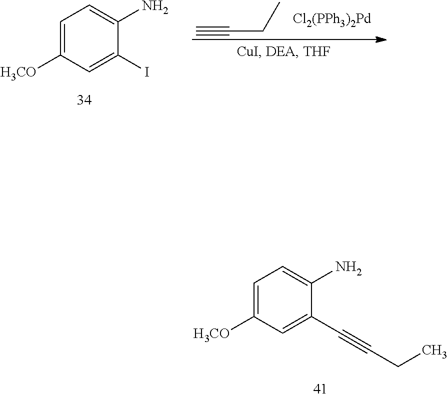

Using a procedure that is substantially similar to the one as described in Example 1, Step 1,2-iodo-4-methoxyphenylamine 34 (0.5215 g, 2.1 mmol) was converted to 41 using 1-butyne. Yield: 0.3262 g (89%), (MS: [M+1=176])

### Step 2:

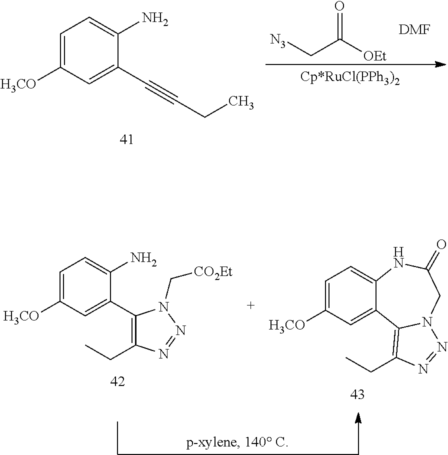

Using a procedure that is substantially similar to the one as described in Example 1, Step 2, compound 41 (321.2 mg, 1.83 mmol) was converted to a mixture of 42 and 43 which was subsequently cyclized to give exclusively 43 as described above. Yield: 193.1 mg (41%) of compound 43 as a solid, (MS: [M+1=259]).

### Step 3:

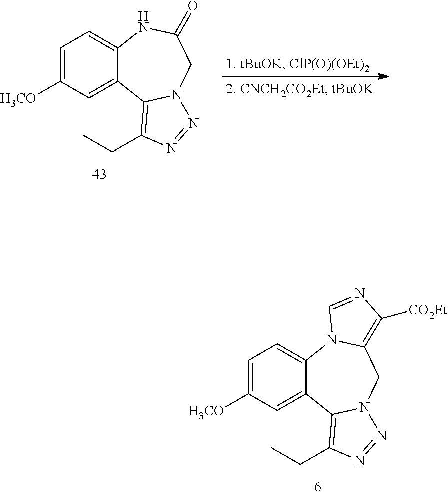 [SMILES: CCc1nnn2c1-c1cc(OC)ccc1NC(=O)C2 ; CCOC(=O)c1ncn2c1Cn1nnc(CC)c1-c1cc(OC)ccc1-2]

Using a procedure that is substantially similar to the one as described in Example 1, Step 3, compound 43 (113 mg, 0.437 mmol) was converted to compound 6. Yield: 29.9 mg (19%), (MS: [M+1=354]).

### Example 7

### Synthesis of Compound 7

### Step 1:

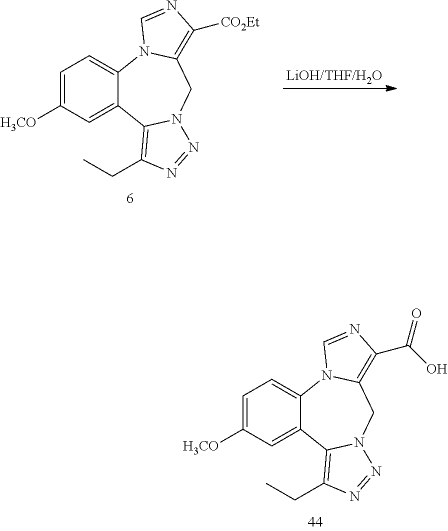 [SMILES: CCOC(=O)c1ncn2c1Cn1nnc(CC)c1-c1cc(OC)ccc1-2 ; CCc1nnn2c1-c1cc(OC)ccc1-n1cnc(C(=O)O)c1C2]

Using a procedure that is substantially similar to the one as described in Example 2, Step 1, compound 6 (52.8 mg, 0.149 mmol) was converted to compound 44. Yield: 31.0 mg (64%), (MS: [M+1=326]).

### Step 2:

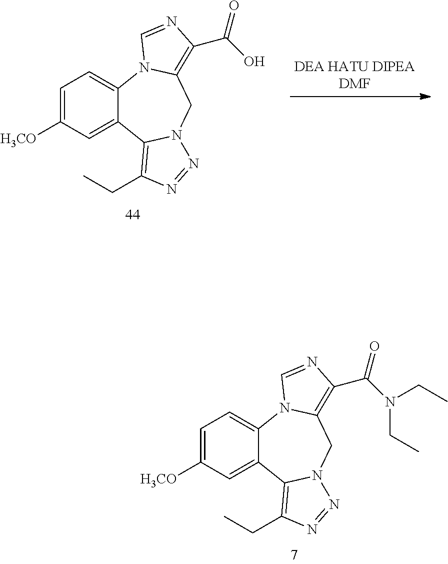 [SMILES: CCc1nnn2c1-c1cc(OC)ccc1-n1cnc(C(=O)O)c1C2 ; CCc1nnn2c1-c1cc(OC)ccc1-n1cnc(C(=O)N(CC)CC)c1C2]

Using a procedure that is substantially similar to the one as described in Example 2, Step 2, compound 44 (32.1 mg, 0.0908 mmol) was converted to compound 7. Yield: 34.7 mg (100%) as a yellow solid, (MS: [M+1=381]).

### Example 8

### Synthesis of Compound 8

### Step 1:

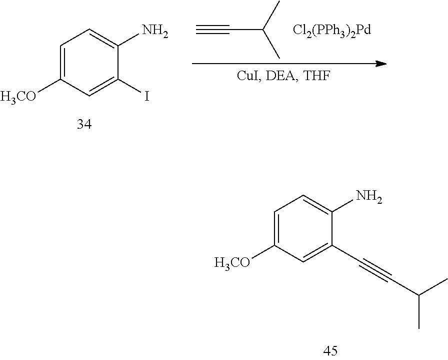

Using a procedure that is substantially similar to the one as described in Example 1, Step 1,2-iodo-4-methoxyphenylamine 34 (0.5 g, 2 mmol) was converted to 45 using 3-methyl-1-butyne. Yield: 0.25 g (38%), (MS: [M+1=190]).

### Step 2:

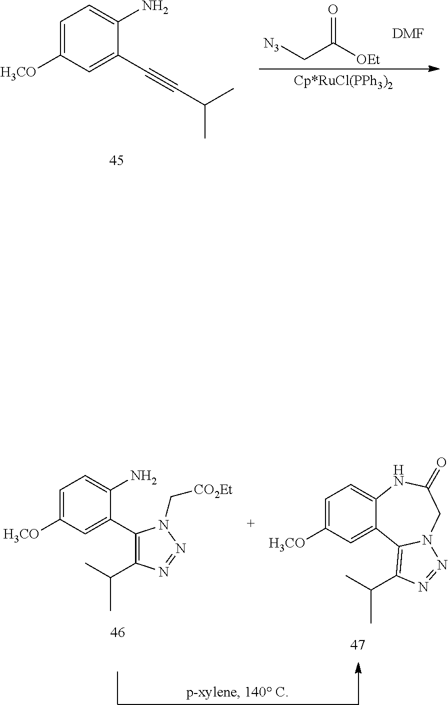

Using a procedure that is substantially similar to the one as described in Example 1, Step 2, compound 45 (130 mg, 0.69 mmol) was converted to a mixture of 46 and 47, which was subsequently cyclized to give exclusively 47 as described above. Yield: 23 mg (12%) of compound 47 as a solid, (MS: [M+1=273]).

### Step 3:

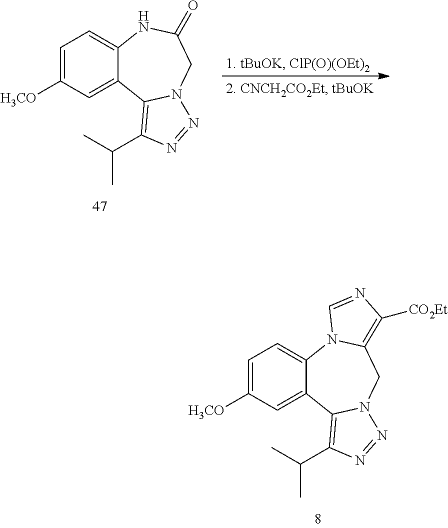 [SMILES: COc1ccc2c(c1)-c1c(C(C)C)nnn1CC(=O)N2 ; CCOC(=O)c1ncn2c1Cn1nnc(C(C)C)c1-c1cc(OC)ccc1-2]

Using a procedure that is substantially similar to the one as described in Example 1, Step 3, compound 47 (23.2 mg, 0.0852 mmol) was converted to compound 8. Yield: 4 mg (13%), (MS: [M+1=368]).

### Example 9

### Synthesis of Compound 9

### Step 1:

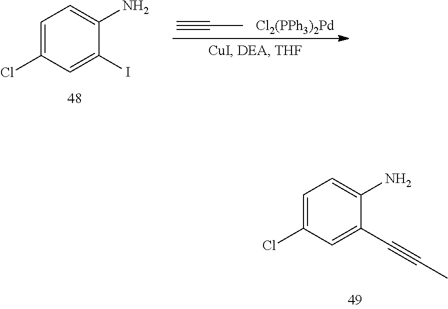

Using a procedure that is substantially similar to the one as described in Example 1, Step 1,2-iodo-4-chlorophenylamine 48 (1 g, 3.95 mmol) was converted to 49 using methyl acetylene. Yield: 0.521 g (80%), (MS: [M+1=166]).

### Step 2:

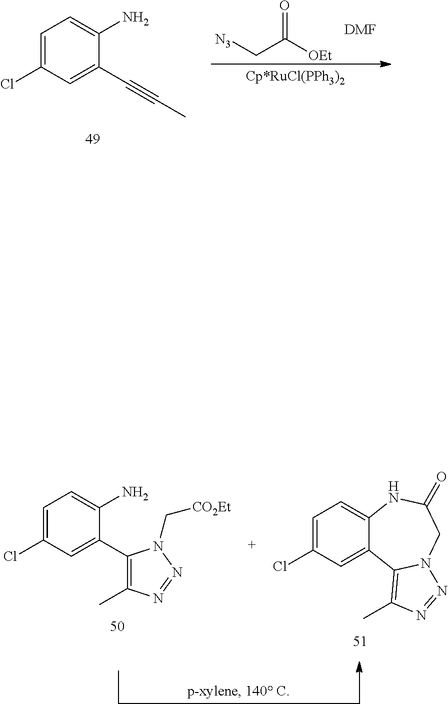

Using a procedure that is substantially similar to the one as described in Example 1, Step 2, compound 49 (514 mg, 3.12 mmol) was converted to a mixture of 50 and 51 which was subsequently cyclized to give exclusively 51 as described above. Yield: 335 mg (43%) of compound 51 as a solid, (MS: [M+1=249]).

### Step 3:

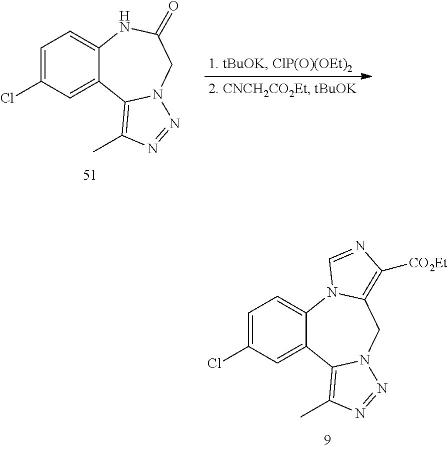 [SMILES: Cc1nnn2c1-c1cc(Cl)ccc1NC(=O)C2 ; CCOC(=O)c1ncn2c1Cn1nnc(C)c1-c1cc(Cl)ccc1-2]

Using a procedure that is substantially similar to the one as described in Example 1, Step 3, compound 51 (187 mg, 0.752 mmol) was converted to compound 9. Yield: 62 mg (24%) as a yellow foam, (MS: [M+1=344]).

### Example 10

### Synthesis of Compound 10

### Step 1:

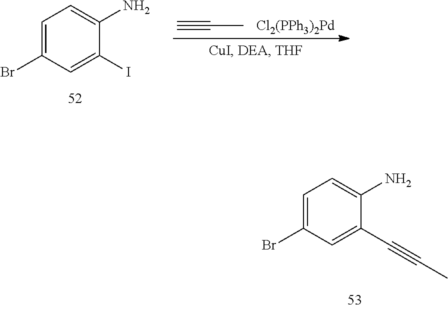

Using a procedure that is substantially similar to the one as described in Example 1, Step 1,2-iodo-4-bromophenylamine 52 (1 g, 3.36 mmol) was converted to 53 using methyl acetylene. Yield: 0.65 g (91%), (MS: [M+1=211]).

### Step 2:

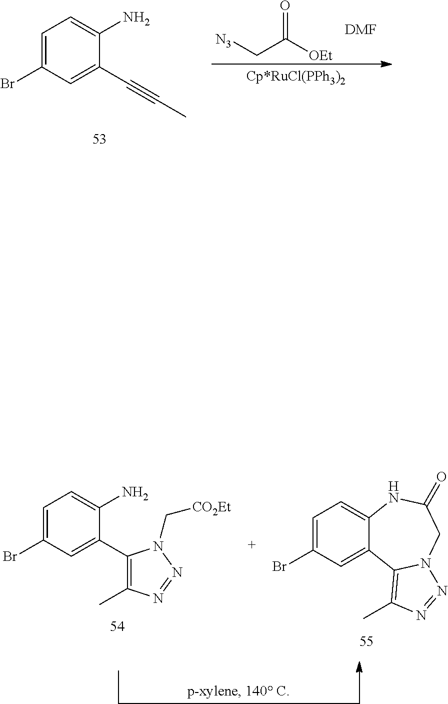

Using a procedure that is substantially similar to the one as described in Example 1, Step 2, compound 53 (673 mg, 3.2 mmol) was converted to a mixture of 54 and 55, which was subsequently cyclized to give exclusively 55 as described above. Yield: 412 mg (44%) of compound 55 as a solid, (MS: [M+1=294]).

### Step 3:

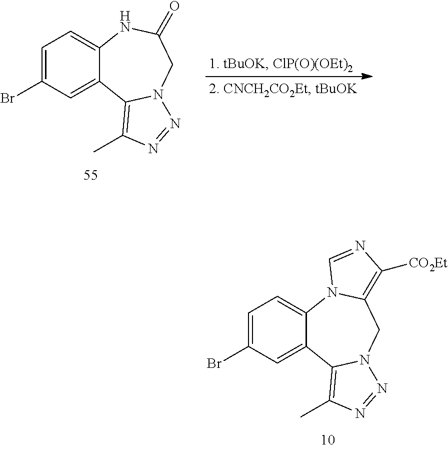 [SMILES: Cc1nnn2c1-c1cc(Br)ccc1NC(=O)C2 ; CCOC(=O)c1ncn2c1Cn1nnc(C)c1-c1cc(Br)ccc1-2]

Using a procedure that is substantially similar to the one as described in Example 1, Step 3, compound 55 (125.8 mg, 0.429 mmol) was converted to compound 10. Yield: 38.8 mg (23%) as a yellow foam, (MS: [M+1=389]).

### Example 11

### Assessing α5-Containing GABA_{A} Receptor (GABA_{A}R) Agonist Activity

Step 1: Establish Clones of GABA_{A}R Subunits (α5, β3, γ2, α1, α2 and α3) and Prepare the Corresponding cRNAs:

Human clones of GABA_{A}-R α5, β3, γ2, α1, α2 and α3 subunits are obtained from commercial resources (e.g., OriGene, http://www.origene.com and Genescript, http://www.genescript.com). These clones are engineered into pRC, pCDM, pcDNA, and pBluescript KSM vector (for oocyte expression) or other equivalent expression vectors. Conventional transfection agents (e.g., FuGene, Lipofectamine 2000, or others) are used to transiently transfect host cells.

Step 2—Functional GABA_{A}R Assay of α5β3γ2, α1β3γ2, α2β3γ2, and α3β3γ2, Subtypes in Xenopus Oocyte Expression System:

cRNAs encoding α5, β3, γ2, α1, α2 and α3 subunits are transcribed in vitro using T3 mMESSAGE mMACHINE Kit (Ambion) and injected (in a ratio of α:β:γ=2:2:1 or other optimized conditions) into oocytes freshly prepared from Xenopus laevis. After two days of culturing, GABA-gated Cl— currents from oocytes are performed using TEVC setups (Warner Instruments, Inc., Foster City, Calif.). GABA, benzodiazepine, and diazepam are used as reference compounds to validate the system.

Step 3—Evaluate Test Compounds for Agonist Activity on the α5β3γ2 Subtype and Test Off-Target Activity on the α1 to α3 Coupled β3γ2 Subtypes when the EC50=5 μM Selectivity Cut-Off is Reached:

The GABA-gated Cl— current from oocytes are measured in the TEVC setup in the presence of the test compounds. The agonist activity of each the test compounds is tested in a 5-point dose-response assay. The test compounds include some reference compounds (literature EC50 values for the α5β3γ2 subtype are in the range of 3-10 μM). EC50s in the α5β3γ2 subtype are obtained for each compound. If the EC50 in α5β3γ2 is ≦5 μM, then the EC50 of the other three subtypes (α1β2γ2, α2β3γ2, and α3β3γ2) is further determined individually in order to test for selectivity of the compounds in the α5β3γ2 subtype over other subtypes.

Step 4—Evaluate Further Test Compounds on the α5β3γ2 Subtype and Test Off-Target Activities when the EC50=0.5 μM Selectivity Cut-Off is Reached:

The second batch of test compounds are tested using the same strategy, but with a lower EC50 cutoff (0.5 μM). Again, the EC50s of the α5β3γ2 subtype for each of the compounds is determined. The α1 to α3 coupled β3γ2 subtypes are tested only if the EC50 for the α5-containing receptor is <0.5 μM.

### Example 12

### Evaluating Compounds for Agonist Activity on the GABA_{A} α5 Receptors

The agonist activity of the compounds of this invention was determined by measuring their effect on GABA-gated Cl— current from Xenopus oocytes expressing GABA_{A} α5β3γ2 subtype receptor in a two-electrode voltage clamp (TEVC) setup. Compounds demonstrating greater than 5% potentiation of the GABA EC₅₀ were indicative of compounds with positive allosteric modulation of the GABA_{A} α5 receptor. That is, these compounds would enhance the effects of GABA at the GABA_{A} α5 receptor.

### Materials

Adult female Xenopus laevis frogs were purchased from Nasco (Fort Atkinson, Wis.). Gentamicin, 3-aminobenzoic acid ethyl ester, GABA, Diazepam, Flumazenil, and collagenase were purchased from Sigma (St. Louis, Mo.). All chemicals used were of reagent grade. GABA stocks were prepared in the extracellular solution, i.e., Modified Barth's Saline (MBS) containing NaCl (88 mM), KCl (2 mM), MgSO₄ (0.82 mM), Ca(NO₃)₂ (0.33 mM), CaCl₂ (0.41 mM), NaHCO₃ (2.4 mM) and HEPES (10 mM). Stock solutions of Diazepam, Flumazenil and compounds of the present invention were prepared in dimethyl sulfoxide (DMSO) and then diluted to an appropriate concentration with the extracellular solution just before use. To avoid adverse effects from DMSO exposure, the final concentration of DMSO was not higher than 0.3% (v/v).

### Experimental Procedures

### (A) Expression of GABA_{A}-R α5β3γ2 or α1β2γ2 subtype in Xenopus Oocytes

Xenopus oocytes were isolated according to previously published procedures (see, e.g., Goldin et al. Methods Enzymol. 207:266-279 (1992)). The isolated Xenopus oocytes were injected with GABA_{A}R cDNAs (1:1:1 ratio for a total volume of 1 ng of α1β2γ2 or α5β3γ2) cloned into mammalian expression vectors. In particular, α1, β2, γ2 were cloned into pcDNA3.1. and α5 and β3 were cloned into pcDNA3.1 myc-His. Vectors were verified by partial sequencing (DNA Core Facility, University of Southern California, USA). After injection, oocytes were stored in incubation medium (Modified Barth's Saline (MBS) supplemented with 2 mM sodium pyruvate, 0.5 mM theophylline and 50 mg/L gentamycin), in petri dishes (VWR, San Dimas, Calif.). All solutions were sterilized by passage through 0.22 μM filters. Oocytes, stored at 18° C., usually expressed GABA_{A}Rs (e.g., α5β3γ2 or α1β2γ2 subtype), 1-2 days after injections. Oocytes were used in experiments for up to 5 days after injection.

### (B) GABA Dose-Response in Xenopus Oocyte Expressing α1 and α5 GABA_{A} Rs

A high-throughput two-electrode voltage clamp (TEVC) system (OpusXpress A6000; Molecular Devices, Union City, Calif.), which automates the impalement of oocytes, fluid delivery and current recording from 8 oocytes in parallel, was used to carry out all electrophysiological recordings.

Xenopus Oocytes expressing GABA_{A}-R α5β3γ2 or α1β2γ2 subtype, as prepared in section (A) above, were placed in 8 chambers of OpusXpress and perfused by MBS at 3 mL/min. Glass electrodes back-filled with 3 M KCl (0.5-3 megaohms) were used. Membrane potential of oocytes was voltage-clamped at −60 mV. Oocytes with holding current larger than 0.5 μA were discarded.

Different concentrations of GABA (3 μM-10 mM for α1-containing GABA_{A}Rs, or 0.3 μM-1 mM for α5-containing GABA_{A}Rs) were applied once for 30 sec, with 5-15 min washes between the applications. Longer wash periods were allowed after the applications of higher GABA concentrations. At the start of each week, a GABA dose-response experiment was conducted to determine an approximate GABA EC₅₀ concentration for the batch of oocytes. EC₅₀ ranged from 100-200 μM for α1-containing GABA_{A}Rs, and 10-20 μM α5-containing GABA_{A}Rs.

### (C) Functional GABA_{A}R Assay of α5β3γ2 or α1β2γ2 Subtype in Xenopus Oocyte Expression System Using Diazepam and Flumazenil as Reference Compounds

Diazepam and Flumazenil were used as reference compounds. In this study the GABA-gated Cl current from oocytes expressing α5β3γ2 GABA_{A}R was measured in the TEVC setup in the presence of Diazepam and Flumazenil. GABA EC₂₀ was applied for 30 sec 4-5 times to establish a stable response. 1 μM Diazepam was pre-applied for 60 sec, followed by co-application of 1 μM Diazepam and GABA at EC₂₀ concentration for 30 sec. After a 15-20 min wash, a combination of 1 μM Diazepam and 10 μM Flumazenil was applied for 60 sec followed by co-application of the same combination with GABA at EC₂₀ concentrations for 30 sec. After a 15-20 min wash, co-application of 1 μM Diazepam and EC₂₀ GABA was repeated to establish the recovery.

The effect of Diazepam was analyzed from the peak amplitude of diazepam-(plus EC₂₀ GABA)-induced current (test 1) with the peak amplitude of GABA-induced current before the diazepam application (reference). The effect of Flumazenil was determined from the peak amplitude of Diazepam-plus-Flumazenil-(plus EC₂₀ GABA)-induced current (test 2) normalized on the peak amplitude of diazepam-induced current (control). Other compounds may also be used in this study as reference compounds. For example, methyl-6,7-dimethoxy-4-ethyl-beta-carboline-3-carboxylate (DMCM) and L655708 were tested at 1 μM, using the same protocol.

### (C) Agonist Activity of Test Compounds on α5β3γ2 Subtype GABA_{A}R

Compounds of the present invention were initially screened at 1 μM for their ability to potentiate an EC₅₀ concentration of GABA in oocytes containing GABA_{A} receptors (α5β3γ2), using a protocol essentially similar to the one presented above for Diazepam and Flumazenil (see section (B)). In this study, the GABA-gated Cl⁻ current from oocytes expressing GABA_{A}R α5β3γ2 subtype was measured in the TEVC setup in the presence of the test compounds. Specifically, GABA EC₅₀ was applied for 30 sec 4-5 times to establish stable response. Next, the test compounds (1 μM) were pre-applied for 60 sec, followed by co-administration of the test compounds (1 μM) and GABA at EC₅₀ concentration for 30 sec. After a 15-20 min wash, EC₅₀ GABA was tested once again. Upon conclusion of compound testing and successful washout, a 1.0 μM diazepam was tested and used for comparative activity on the two GABA_{A}R subtypes.

The effect of each test compound was determined from the peak amplitude of Diazepam-plus-compound-(plus EC₅₀ GABA)-induced current normalized on the peak amplitude of Diazepam-(plus EC₅₀ GABA)-induced current (control). Other concentrations of the test compound may also be tested following the same protocol.

A compound which demonstrates greater than 5% potentiation of the GABA EC₅₀ is indicative that the compound has a positive allosteric modulatory effect on the GABA_{A} α5 receptor. Such compound will enhance the effects of GABA at the GABA_{A} α5 receptor. Exemplary compounds that demonstrated greater than 5% potentiation of the GABA EC₅₀ are shown in Table 1 below.

TABLE 1
Exemplary compounds with >5% Potentiation of GABA EC50
Concentration in Oocytes containing GABA_{A} receptors (α5β3γ2)

| | | Compound | |
|---|---|---|---|
| | Chemical Name | Concentration | GABA α5 EC50 |
| Compound | and Structure | in μM | % potentiation |
| 2 | 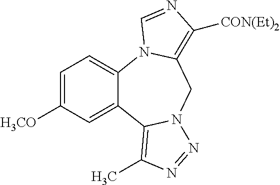 | 10 | 6.2 |
| 7 | 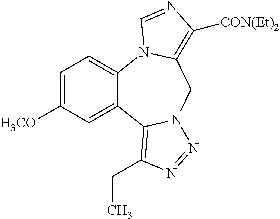 | 1 | 11.1 |
| 9 | 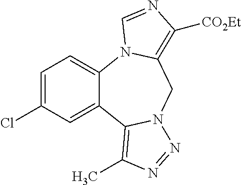 [SMILES: CCOC(=O)c1ncn2c1Cn1nnc(C)c1-c1cc(Cl)ccc1-2] | 1 | 8.3 |

### (D) Evaluate Test Compounds for Off-Target Activity on the α1β2γ2 Subtype

Compounds having a positive allosteric modulatory effect on GABA_{A} α5 receptors will be evaluated across a range of concentrations (i.e., at 0.01, 0.1, 1, 10 and 100 μM) to determine the concentration response curve at GABA_{A} α5 receptors (α5β3γ2) and selectivity vs. GABA_{A} al receptors (α1β2γ2).

### (E) Data Analysis

Data for each experimental point were obtained from 4 or more Xenopus oocytes and from at least two different frogs. The n refers to the number of Xenopus oocytes tested. Results are expressed as mean±SEM. Where no error bars are shown, they are smaller than the symbols. Prism (GraphPAD Software, San Diego, Calif.) and Excel were used to perform curve fitting and statistical analyses. GABA concentration response curves were generated using non-linear regression analysis: [I=Iₘₐₓ [A]^{nH}/([A]^{nH}+EC₅₀^{nH})] where I is the peak current recorded following application of a range of agonist concentrations, [A]; Iₘₐₓ is the estimated maximum current; EC₅₀ is the GABA concentration required for a half-maximal response and n_{H} is the Hill slope.

### Example 13

### Effect of Methyl 3,5-diphenylpyridazine-4-carboxylate in Aged-Impaired (AI) Rats

Methyl 3,5-diphenylpyridazine-4-carboxylate, corresponding to compound number 6 in van Niel et al. J. Med. Chem. 48:6004-6011 (2005), is a selective α5-containing GABA_{A} R agonist. It has an α5 in vitro efficacy of +27 (EC₂₀). The effect of methyl 3,5-diphenylpyridazine-4-carboxylate in aged-impaired rats was studied using a RAM task. Moreover, receptor occupancy by methyl 3,5-diphenylpyridazine-4-carboxylate in α5-containing GABA_{A} receptor was also studied.

(A) Effect of Methyl 3,5-diphenylpyridazine-4-carboxylate in Aged-Impaired Rats Using a Radial Arm Maze (RAM) Behavioral Task

The effects of methyl 3,5-diphenylpyridazine-4-carboxylate on the in vivo spatial memory retention of aged-impaired (AI) rats were assessed in a Radial Arm Maze (RAM) behavioral task using vehicle control and four different dosage levels of methyl 3,5-diphenylpyridazine-4-carboxylate (0.1 mg/kg, 0.3 mg/kg, 1 mg/kg and 3 mg/kg, ip). RAM behavioral tasks were performed on eight AI rats. All five treatment conditions (vehicle and four dosage levels) were tested on all eight rats.

The RAM apparatus used consisted of eight equidistantly-spaced arms. An elevated maze arm (7 cm width×75 cm length) projected from each facet of an octagonal center platform (30 cm diameter, 51.5 cm height). Clear side walls on the arms were 10 cm high and were angled at 65° to form a trough. A food well (4 cm diameter, 2 cm deep) was located at the distal end of each arm. Froot Loops™ (Kellogg Company) were used as rewards. Blocks constructed of Plexiglas™ (30 cm height×12 cm width) could be positioned to prevent entry to any arm. Numerous extra maze cues surrounding the apparatus were also provided.

The AI rats were initially subjected to a pre-training test (Chappell et al. Neuropharmacology 37: 481-487, 1998). The pre-training test consisted of a habituation phase (4 days), a training phase on the standard win-shift task (18 days) and another training phase (14 days) in which a brief delay was imposed between presentation of a subset of arms designated by the experimenter (e.g., 5 arms available and 3 arms blocked) and completion of the eight-arm win-shift task (i.e., with all eight arms available).

In the habituation phase, rats were familiarized to the maze for an 8-minute session on four consecutive days. In each of these sessions, food rewards were scattered on the RAM, initially on the center platform and arms and then progressively confined to the arms. After this habituation phase, a standard training protocol was used, in which a food pellet was located at the end of each arm. Rats received one trial each day for 18 days. Each daily trial terminated when all eight food pellets had been obtained or when either 16 choices were made or 15 minutes had elapsed. After completion of this training phase, a second training phase was carried out in which the memory demand was increased by imposing a brief delay during the trial. At the beginning of each trial, three arms of the eight-arm maze were blocked. Rats were allowed to obtain food on the five arms to which access was permitted during this initial “information phase” of the trial. Rats were then removed from the maze for 60 seconds, during which time the barriers on the maze were removed, thus allowing access to all eight arms. Rats were then placed back onto the center platform and allowed to obtain the remaining food rewards during this “retention test” phase of the trial. The identity and configuration of the blocked arms varied across trials.

The number of “errors” the AI rats made during the retention test phase was tracked. An error occurred in the trial if the rats entered an arm from which food had already been retrieved in the pre-delay component of the trial, or if the rat re-visited an arm in the post-delay session that it had already visited.

After completion of the pre-training test, rats were subjected to trials with more extended delay intervals, i.e., a two-hour delay, between the information phase (presentation with some blocked arms) and the retention test (presentation of all arms). During the delay interval, rats remained off to the side of the maze in the testing room, on carts in their individual home cages. AI rats were pretreated 30-40 minutes before daily trials with a one-time shot of the following five conditions: 1) vehicle control—5% dimethyl sulfoxide, 25% polyethylene glycol 300 and 70% distilled water; 2) methyl 3,5-diphenylpyridazine-4-carboxylate at 0.1 mg/kg; 3) methyl 3,5-diphenylpyridazine-4-carboxylate at 0.3 mg/kg; 4) methyl 3,5-diphenylpyridazine-4-carboxylate at 1 mg/kg); and 5) methyl 3,5-diphenylpyridazine-4-carboxylate at 3 mg/kg; through intraperitoneal (i.p.) injection. Injections were given every other day with intervening washout days. Each AI rat was treated with all five conditions within the testing period. To counterbalance any potential bias, drug effect was assessed using ascending-descending dose series, i.e., the dose series was given first in an ascending order and then repeated in a descending order. Therefore, each dose had two determinations.

Parametric statistics (paired t-tests) was used to compare the retention test performance of the AI rats in the two-hour delay version of the RAM task in the context of different doses of methyl 3,5-diphenylpyridazine-4-carboxylate and vehicle control (see FIG. 1). The average numbers of errors that occurred in the trials were significantly fewer with methyl 3,5-diphenylpyridazine-4-carboxylate treatment of 3 mg/kg (average no. of errors±standard error of the mean (SEM)=1.31±0.40) than using vehicle control (average no. of errors±SEM=3.13±0.62). Relative to vehicle control treatment, methyl 3,5-diphenylpyridazine-4-carboxylate significantly improved memory performance at 3 mg/kg (t(7)=4.233, p=0.004).

The therapeutic dose of 3 mg/kg became ineffective when the AI rats were concurrently treated with 0.3 mg/kg of TB21007, a α5-containing GABA_{A} R inverse agonist. The average numbers of errors made by rats with the combined TB21007/methyl 3,5-diphenylpyridazine-4-carboxylate treatment (0.3 mg/kg TB21007 with 3 mg/kg methyl 3,5-diphenylpyridazine-4-carboxylate) was 2.88±1.32, and was no different from rats treated with vehicle control (3.13±1.17 average errors). Thus, the effect of methyl 3,5-diphenylpyridazine-4-carboxylate on spatial memory is a GABA_{A} α5 receptor-dependent effect (see FIG. 1).

(B) Effect of Methyl 3,5-diphenylpyridazine-4-carboxylate on α5-Containing GABA_{A} Receptor Occupancy

### Animals

Adult male Long Evans rats (265-295 g, Charles River, Portage, Mich., n=4/group) were used for GABA_{A}α5 receptor occupancy studies. Rats were individually housed in ventilated stainless-steel racks on a 12:12 light/dark cycle. Food and water were available ad libitum. In additional studies to evaluate compound exposures at behaviorally active doses, young or aged Long Evan rats (n=2-4/group) were used for these studies.

### Compounds

Ro 15-4513 was used as a receptor occupancy (RO) tracer for GABA_{A}α5 receptor sites in the hippocampus and cerebellum. Ro 15-4513 was chosen as the tracer based on its selectivity for GABA_{A}α5 receptors relative to other alpha subunit containing GABA_{A} receptors and because it has been successfully used for GABA_{A}α5 RO studies in animals and humans (see, e.g., Lingford-Hughes et al., J. Cereb. Blood Flow Metab. 22:878-89 (2002); Pym et al, Br. J. Pharmacol. 146: 817-825 (2005); and Maeda et al., Synapse 47: 200-208 (2003)). Ro 15-4513 (1 μg/kg), was dissolved in 25% hydroxyl-propyl beta-cyclodextrin and administered i.v. 20′ prior to the RO evaluations. Methyl 3,5-diphenylpyridazine-4-carboxylate (0.1-10 mg/kg) was synthesized by Nox Pharmaceuticals (India) and was dissolved in 25% hydroxyl-propyl beta-cyclodextrin and administered i.v. 15′ prior to tracer injection. Compounds were administered in a volume of 0.5 ml/kg except for the highest dose of methyl 3,5-diphenylpyridazine-4-carboxylate (10 mg/kg) which was administered in a volume of 1 ml/kg due to solubility limitations.

### Tissue Preparation and Analysis

The rats were sacrificed by cervical dislocation 20′ post tracer injection. The whole brain was rapidly removed, and lightly rinsed with sterile water. Trunk blood was collected in EDTA coated eppendorf tubes and stored on wet ice until study completion. Hippocampus and cerebellum were dissected and stored in 1.5 ml eppendorf tubes, and placed on wet ice until tissue extraction. In a drug naïve rat, six cortical brain tissues samples were collected for use in generating blank and standard curve samples.

Acetonitrile containing 0.1% formic acid was added to each sample at a volume of four times the weight of the tissue sample. For the standard curve (0.1-30 ng/g) samples, a calculated volume of standard reduced the volume of acetonitrile. The sample was homogenized (FastPrep-24, Lysing Matrix D; 5.5 m/s, for 60 seconds or 7-8 watts power using sonic probe dismembrator; Fisher Scientific) and centrifuged for 16-minutes at 14,000 rpm. The (100 μl) supernatant solution was diluted by 300 μl of sterile water (pH 6.5). This solution was then mixed thoroughly and analyzed via LC/MS/MS for R δ 15-4513 (tracer) and methyl 3,5-diphenylpyridazine-4-carboxylate.

For plasma exposures, blood samples were centrifuged at 14000 rpm for 16 minutes. After centrifuging, 50 ul of supernatant (plasma) from each sample was added to 200 μl of acetonitrile plus 0.1% formic acid. For standard curve (1-1000 ng/ml) samples, a calculated volume of standard reduced the volume of acetonitrile. Samples were sonicated for 5 minutes in an ultrasonic water bath, followed by centrifugation for 30 minutes, at 16000 RPM. 100 ul of supernatant was removed from each sample vial and placed in a new glass auto sample vial, followed by the addition of 300 μl of sterile water (pH 6.5). This solution was then mixed thoroughly and analyzed via LC/MS/MS for methyl 3,5-diphenylpyridazine-4-carboxylate.

Receptor occupancy was determined by the ratio method which compared occupancy in the hippocampus (a region of high GABA_{A}α5 receptor density) with occupancy in the cerebellum (a region with low GABA_{A}α5 receptor density) and additionally by a high dose of the GABA_{A}α5 negative allosteric modulator L-655,708 (10 mg/kg, i.v.) to define full occupancy.

Vehicle administration followed by tracer administration of 1 μg/kg, i.v., of Ro 15-4513 resulted in >5-fold higher levels of Ro 15-4513 in hippocampus (1.93±0.05 ng/g) compared with cerebellum (0.36±0.02 ng/g). Methyl 3,5-diphenylpyridazine-4-carboxylate (0.01-10 mg/kg, i.v.) dose-dependently reduced Ro 15-4513 binding in hippocampus, without affecting cerebellum levels of Ro 15-4513 (FIG. 2) with a dose of 10 mg/kg, i.v., demonstrating >90% occupancy (FIG. 3). Both methods of calculating RO yielding very similar results with ED50 values for methyl 3,5-diphenylpyridazine-4-carboxylate as 1.8 mg/kg or 1.1 mg/kg based on the ratio method or using L-755,608 to define occupancy.

Methyl 3,5-diphenylpyridazine-4-carboxylate exposure was below the quantification limits (BQL) at 0.01 mg/kg, i.v., in both plasma and hippocampus and but was detectable at low levels in hippocampus at 0.1 mg/kg, i.v. (see Table 2). Hippocampal exposure was linear as a 10-fold increase in dose from 0.1 to 1 mg/kg, i.v., resulted in a 12-fold increase in exposure. Increasing the dose from 1 to 10 mg/kg, i.v., only increased the exposure by ˜5-fold. Plasma exposure increased 12-fold as the dose increased from 1 to 10 mg/kg, i.v.

TABLE 2
% GABA_{A} α5 Receptor Occupancy by methyl 3,5-diphenylpyridazine-
4-carboxylate (0.01-10 mg/kg, i.v.). Hippocampus and Plasma
Exposure of methyl 3,5-diphenylpyridazine-4-carboxylate
by Treatment Group in young Long Evans rats.

| | % RO | % RO | | |
|---|---|---|---|---|
| Dose | (L-655,708 | (Ratio | Plasma | Hippocampus |
| (mg/kg, | Method) | Method) | ng/mL | ng/g |
| i.v.) | (SEM) | (SEM) | (SEM) | (SEM) |
| 0.01 | 19.2 (11.1) | 15.7 (9.1) | BQL | BQL |
| 0.1 | 16.4 (4.9) | 13.4 (4.0) | BQL | 14.6 (3.5) |
| 1 | 38.5 (11.2) | 31.5 (9.1) | 62.8 (6.1) | 180.0 (10.3) |
| 10 | 110.0 (6.6) | 90.2 (5.4) | 763.5 (85.7) | 947.2 (51.3) |

Additional studies were conducted in aged Long-Evans rats in order to determine the exposures at the behaviorally relevant doses in the cognition studies. Exposure in young Long-Evans rats was also determined to bridge with the receptor occupancy studies that were conducted in young Long-Evans rats. Exposures in young and aged Long-Evans rats were relatively similar (Table 3, FIG. 4). Increasing the dose 3-fold from 1 to 3 mg/kg, ip resulted in a greater than dose-proportional increase in exposure in young and aged rats in both hippocampus and plasma with increases ranging from 4.5 to 6.6-fold.

TABLE 3
Hippocampus and Plasma Exposure of methyl 3,5-diphenylpyridazine-
4-carboxylate in Young Long Evans Rats by Treatment Group

| | Young | Young | Aged | Aged |
|---|---|---|---|---|
| Dose | Hippocampus | Plasma | Hippocampus | Plasma |
| (mg/kg, | ng/g | ng/mL | ng/g | ng/mL |
| ip) | (SEM) | (SEM) | (SEM) | (SEM) |
| 1 | 25.9 (1.7) | 20.0 (1.4) | 38.8 (21.7) | 45.2 (29.6) |
| 3 | 129.1 (22.4) | 132.9 (19.5) | 177.5 (19.5) | 196 (18.2) |

In the RO studies, an exposure of 180 ng/g in hippocampus (1 mg/kg, i.v.) represented 32-39% receptor occupancy depending on method used to determine RO. This exposure is comparable to that observed in aged rats at 3 mg/kg, i.p., suggesting that 30-40% RO is required for cognitive efficacy in this model.

These studies demonstrated that methyl 3,5-diphenylpyridazine-4-carboxylate produced dose-dependent increase in GABA_{A} α5 receptor occupancy. Methyl 3,5-diphenylpyridazine-4-carboxylate also demonstrated good brain exposure with brain/plasma ratios>1. The studies further demonstrated that methyl 3,5-diphenylpyridazine-4-carboxylate was producing its cognitive enhancing effects by positive allosteric modulation at the GABA_{A} α5 subtype receptor.

### Example 14

### Effect of Ethyl 3-methoxy-7-methyl-9H-benzo[f]imidazo[1,5-a][1,2,4]triazolo[4,3-d][1,4]diazepine-10-carboxylate in Aged-Impaired (AI) Rats

Ethyl 3-methoxy-7-methyl-9H-benzo[f]imidazo[1,5-a][1,2,4]triazolo[4,3-d][1,4]diazepine-10-carboxylate, corresponding to compound number 49 in Achermann et al. Bioorg. Med. Chem. Lett., 19:5746-5752 (2009), is a selective α5-containing GABA_{A} R agonist.

The effect of ethyl 3-methoxy-7-methyl-9H-benzo Wimidazo[1,5-a][1,2,4]triazolo[4,3-d][1,4]diazepine-10-carboxylate on the in vivo spatial memory retention of aged-impaired (AI) rats was assessed in a Radial Arm Maze (RAM) behavioral task that is essentially similar to the task as described in Example 3 (A), using vehicle control (25% cyclodextrin, which was tested 3 times: at the beginning, middle and end of ascending/descending series) and six different doses levels (0.1 mg/kg, 0.3 mg/kg, 1 mg/kg, 3 mg/kg, 10 mg/kg and 30 mg/kg, each dose was tested twice) of ethyl 3-methoxy-7-methyl-9H-benzo[f]imidazo[1,5-a][1,2,4]triazolo[4,3-d][1,4]diazepine-10-carboxylate. The same experiment was repeated using the same vehicle control and doses of ethyl 3-methoxy-7-methyl-9H-benzo[f]imidazo[1,5-a][1,2,4]triazolo[4,3-d][1,4]diazepine-10-carboxylate, where the vehicle control was tested 5 times, the 3 mg/kg dose of ethyl 3-methoxy-7-methyl-9H-benzo[f]imidazo[1,5-a][1,2,4]triazolo[4,3-d][1,4]diazepine-10-carboxylate was tested 4 times, and the other doses of ethyl 3-methoxy-7-methyl-9H-benzo[f]imidazo[1,5-a][1,2,4]triazolo[4,3-d][1,4]diazepine-10-carboxylate were tested twice.

Parametric statistics (paired t-tests) was used to compare the retention test performance of the AI rats in the four-hour delay version of the RAM task in the context of different doses of ethyl 3-methoxy-7-methyl-9H-benzo[f]imidazo[1,5a][1,2,4]triazolo[4,3-d][1,4]diazepine-10-carboxylate and vehicle control (see FIGS. 5(A) and 5(B)). Relative to vehicle control treatment, ethyl 3-methoxy-7-methyl-9H-benzo[f]imidazo[1,5-a][1,2,4]triazolo[4,3-d][1,4]diazepine-10-carboxylate significantly improved memory performance at 3 mg/kg (t(7)=4.13, p=0.004, or t(7)=3.08, p=0.018) and at 10 mg/kg (t(7)=2.82, p=0.026).

The effect of ethyl 3-methoxy-7-methyl-9H-benzo[f]imidazo[1,5-a][1,2,4]triazolo[4,3-d][1,4]diazepine-10-carboxylate on α5-containing GABA_{A} receptor occupancy was also studied following a procedure that is essentially similar to the one as described in Example 13(B) (see above). This study demonstrated that ethyl 3-methoxy-7-methyl-9H-benzo[f]imidazo[1,5-a][1,2,4]triazolo[4,3-d][1,4]diazepine-10-carboxylate (0.01-10 mg/kg, i.v.) reduced Ro 15-4513 binding in hippocampus, without affecting cerebellum levels of Ro 15-4513 (FIG. 6) with a dose of 10 mg/kg, i.v., demonstrating >90% occupancy (FIG. 7).

### Example 15

### Effect of 6,6 dimethyl-3-(3-hydroxypropyl)thio-1-(thiazol-2-yl)-6,7-dihydro-2-benzothiophen-4(5H)-one in Aged-Impaired Rats Using a Morris Water Maze Behavioral Task

6,6 dimethyl-3-(3-hydroxypropyl)thio-1-(thiazol-2-yl)-6,7-dihydro-2-benzothiophen-4(5H)-one, corresponding to compound 44 in Chambers et al. J. Med. Chem. 46:2227-2240 (2003) is a selective α5-containing GABA_{A} R agonist.

The effects of 6,6 dimethyl-3-(3-hydroxypropyl)thio-1-(thiazol-2-yl)-6,7-dihydro-2-benzothiophen-4(5H)-one on the in vivo spatial memory retention of aged-impaired (AI) rats were assessed in a Morris water maze behavioral task. A water maze is a pool surrounded with a novel set of patterns relative to the maze. The training protocol for the water maze may be based on a modified water maze task that has been shown to be hippocampal-dependent (de Hoz et al., Eur. J. Neurosci., 22:745-54, 2005; Steele and Morris, Hippocampus 9:118-36, 1999).

Cognitively impaired aged rats were implanted unilaterally with a cannula into the lateral ventricle. Stereotaxic coordinates were 1.0 mm posterior to bregma, 1.5 mm lateral to midline, and 3.5 mm ventral to the skull surface. After about a week of recovery, the rats were pre-trained in a water maze for 2 days (6 trials per day) to locate a submerged escape platform hidden underneath the surface of the pool, in which the escape platform location varied from day to day. No intracerebroventricular (ICV) infusion was given during pre-training

After pre-training, rats received ICV infusion of either 100 μg 6,6 dimethyl-3-(3-hydroxypropyl)thio-1-(thiazol-2-yl)-6,7-dihydro-2-benzothiophen-4(5H)-one (n=6) in 5 μl DMSO or vehicle DMSO (n=5) 40 min prior to water maze training and testing. Training consisted of 8 trials per day for 2 days where the hidden escape platform remained in the same location. Rats were given 60 seconds to locate the platform with a 60 seconds inter-trial interval. The rats were given a probe test (120 seconds) 24 hr after the end of training where the escape platform was removed. During the training, there were 4 blocks, where each block had 4 training trials.

Rats treated with vehicle and 6,6 dimethyl-3-(3-hydroxypropyl)thio-1-(thiazol-2-yl)-6,7-dihydro-2-benzothiophen-4(5H)-one found the escape platform about the same time at the beginning of training (block 1). In this block of training, rats treated with vehicle and 6,6 dimethyl-3-(3-hydroxypropyl)thio-1-(thiazol-2-yl)-6,7-dihydro-2-benzothiophen-4(5H)-one both spent about 24 seconds to find the escape platform. However, rats treated with 6,6 dimethyl-3-(3-hydroxypropyl)thio-1-(thiazol-2-yl)-6,7-dihydro-2-benzothiophen-4(5H)-one were able to find the platform more proficiently (i.e., quicker) at the end of training (block 4) than those treated with vehicle alone. In block 4, rats treated with 6,6 dimethyl-3-(3-hydroxypropyl)thio-1-(thiazol-2-yl)-6,7-dihydro-2-benzothiophen-4(5H)-one spent about 9.6 seconds to find the escape platform, while rats treated with vehicle spent about 19.69 seconds. These results suggest that 6,6 dimethyl-3-(3-hydroxypropyl)thio-1-(thiazol-2-yl)-6,7-dihydro-2-benzothiophen-4(5H)-one improved the learning of the water maze task in rats (see FIG. 8(A)).

During a test trial 24 hr after training, the escape platform was removed. The search/swim pattern of the rats was used to measure whether the rats remember where the escape platform was located during pre-trial training in order to test for the long-term memory of the rats. In this trial, “target annulus” is a designated area 1.5 times the size of the escape platform around the area where the platform was located during pre-trial training “Opposite annulus” is a control area of the same size as the size of the target annulus, which is located opposite to the target annulus in the pool. If the rats had good long term memory, they would tend to search in the area surrounding the location where the platform was during the pre-trial training (i.e., the “target” annulus; and not the “opposite” annulus). “Time in annulus” is the amount of time in seconds that the rat spent in the target or opposite annulus area. “Number (#) of crossings” in annulus is the number of times the rat swam across the target or opposite annulus area.

Rats received vehicle spent the same amount of time in the target annulus and opposite annulus, indicating that these rats did not seem to remember where the platform was during the pre-trial training. By contrast, rats treated with 6,6 dimethyl-3-(3-hydroxypropyl)thio-1-(thiazol-2-yl)-6,7-dihydro-2-benzothiophen-4(5H)-one spent significantly more time in the target annulus, and crossed the “target annulus” more often, as compared to the time they spent in, or the number of times they crossed the “opposite annulus”. These results suggest that 6,6 dimethyl-3-(3-hydroxypropyl)thio-1-(thiazol-2-yl)-6,7-dihydro-2-benzothiophen-4(5H)-one improved the long-term memory of rats in the water maze task (see, FIGS. 8(B) and 8(C)).

Compounds of the present invention demonstrated positive allosteric modulatory effect on the GABA_{A} α5 receptor (See, e.g., Example 12). These compounds will enhance the effects of GABA at the GABA_{A} α5 receptor. Therefore, compounds of the present invention should produce cognitive enhancing effects in aged-impaired animals (such as rats), similar to the effects produced by other GABA_{A} α5 receptor selective agonists, such as methyl 3,5-diphenylpyridazine-4-carboxylate, ethyl 3-methoxy-7-methyl-9H-benzo[f]imidazo[1,5-a][1,2,4]triazolo[4,3-d][1,4]diazepine-10-carboxylate, and 6,6 dimethyl-3-(3-hydroxypropyl)thio-1-(thiazol-2-yl)-6,7-dihydro-2-benzothiophen-4(5H)-one (See, e.g., Examples 13-15).

1. A compound of formula I: 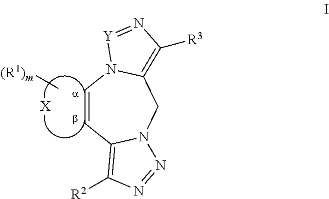
or a pharmaceutically acceptable salt thereof, wherein:
X and the two carbon atoms designated by α and β together form a C5-C10 aromatic ring having 0-4 heteroatoms independently selected from N, O and S;
Y is —N═ or —C(R⁴)═;
m is an integer selected from 0-4;
each occurrence of R¹, R², R³ and R⁴ is independently selected from:
halogen, —R, —OR, —NO₂, —NCS, —CN, —CF₃, —OCF₃, —SiR₃₅—N(R)₂, —SR, —SOR, —SO₂R, —SO₂N(R)₂, —SO₃R, —(CR₂)₁₋₃R, —(CR₂)₁₋₃—OR, —(CR₂)₀₋₃—C(O)NR(CR₂)₀₋₃R, —(CR₂)₀₋₃—C(O)NR(CR₂)₀₋₃OR, —C(O)R, —C(O)C(O)R, —C(O)CH₂C(O)R, —C(S)R, —C(S)OR, —C(O)OR, —C(O)C(O)OR, —C(O)C(O)N(R)₂, —OC(O)R, —C(O)N(R)₂, —OC(O)N(R)₂, —C(S)N(R)₂, —(CR₂)₀₋₃NHC(O)R, —N(R)N(R)COR, —N(R)N(R)C(O)OR, —N(R)N(R)CON(R)₂, —N(R)SO₂R, —N(R)SO₂N(R)₂₅—N(R)C(O)OR, —N(R)C(O)R, —N(R)C(S)R, —N(R)C(O)N(R)₂, —N(R)C(S)N(R)₂, —N(COR)COR, —N(OR)R, —C(═NH)N(R)₂, —C(O)N(OR)R, —C(═NOR)R, —OP(O)(OR)₂, —P(O)(R)₂, —P(O)(OR)₂, and —P(O)(H)(OR);
each R is independently selected from:
H—,
(C1-C12)-aliphatic-,
(C3-C10)-cycloalkyl-,
(C3-C10)-cycloalkenyl-,
[(C3-C10)-cycloalkyl]-(C1-C12)-aliphatic-,
[(C3-C10)-cycloalkenyl]-(C1-C12)-aliphatic-,
(C6-C10)-aryl-,
(C6-C10)-aryl-(C1-C12)aliphatic-,
(C3-C10)-heterocyclyl-,
(C6-C10)-heterocyclyl-(C1-C12)aliphatic-,
(C5-C10)-heteroaryl-, and
(C5-C10)-heteroaryl-(C1-C12)-aliphatic-;
or when two R groups bound to the same atom, the two R groups may be taken together with the atom to which they are bound to form a 3- to 10-membered aromatic or non-aromatic ring having 0-3 heteroatoms independently selected from N, O, S, SO, and SO₂, wherein said ring is optionally fused to a (C6-C10)aryl, (C5-C10)heteroaryl, (C3-C10)cycloalkyl, or a (C3-C10)heterocyclyl;
wherein each occurrence of R is independently substituted with 0-5 R′;
wherein each occurrence of R′ is independently selected from H, halogen, —R″, —OR″, —NO₂, —NCS, —CN, —CF₃, —OCF₃ and —N(R″)₂;
wherein R″ is H or —(C1-C4)-aliphatic;
provided that said compound of formula I is not:

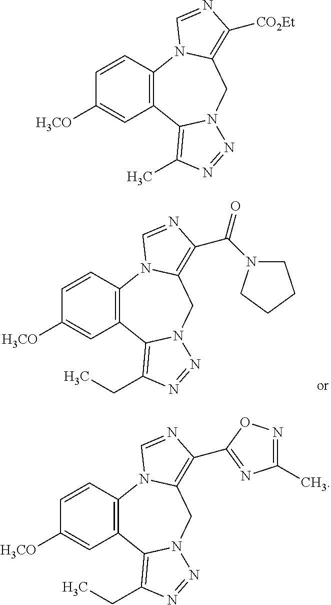

2. A compound of formula II: 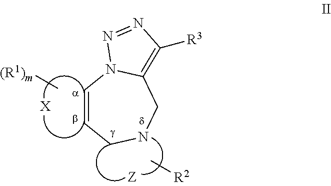
or a pharmaceutically acceptable salt thereof, wherein:
X and the two carbon atoms designated by α and β together form a C5-C10 aromatic ring having 0-4 heteroatoms independently selected from N, O and S;
Z and the carbon atom designated by γ and the N atom designated by δ together form a triazolo ring selected from: 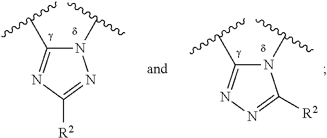
m is an integer selected from 0-4;
each occurrence of R¹, R² and R³ is independently selected from:
halogen, —R, —OR, —NO₂, —NCS, —CN, —CF₃, —OCF₃, —SiR₃, —N(R)₂, —SR, —SOR, —SO₂R, —SO₂N(R)₂, —SO₃R, —(CR₂)₁₋₃R, —(CR₂)₁₋₃—OR, —(CR₂)₀₋₃—C(O)NR(CR₂)₀₋₃R, —(CR₂)₀₋₃—C(O)NR(CR₂)₀₋₃OR, —C(O)R, —C(O)C(O)R, —C(O)CH₂C(O)R, —C(S)R, —C(S)OR, —C(O)OR, —C(O)C(O)OR, —C(O)C(O)N(R)₂, —OC(O)R, —C(O)N(R)₂, —OC(O)N(R)₂, —C(S)N(R)₂, —(CR₂)₀₋₃NHC(O)R, —N(R)N(R)COR, —N(R)N(R)C(O)OR, —N(R)N(R)CON(R)₂, —N(R)SO₂R, —N(R)SO₂N(R)₂, —N(R)C(O)OR, —N(R)C(O)R, —N(R)C(S)R, —N(R)C(O)N(R)₂, —N(R)C(S)N(R)₂, —N(COR)COR, —N(OR)R, —C(═NH)N(R)₂, —C(O)N(OR)R, —C(═NOR)R, —OP(O)(OR)₂, —P(O)(R)₂, —P(O)(OR)₂, and —P(O)(H)(OR);
each R is independently selected from:
H—,
(C1-C12)-aliphatic-,
(C3-C10)-cycloalkyl-,
(C3-C10)-cycloalkenyl-,
[(C3-C10)-cycloalkyl]-(C1-C12)-aliphatic-,
[(C3-C10)-cycloalkenyl]-(C1-C12)-aliphatic-,
(C6-C10)-aryl-,
(C6-C10)-aryl-(C1-C12)aliphatic-,
(C3-C10)-heterocyclyl-,
(C6-C10)-heterocyclyl-(C1-C12)aliphatic-,
(C5-C10)-heteroaryl-, and
(C5-C10)-heteroaryl-(C1-C12)-aliphatic-;
or when two R groups bound to the same atom, the two R groups may be taken together with the atom to which they are bound to form a 3- to 10-membered aromatic or non-aromatic ring having 0-3 heteroatoms independently selected from N, O, S, SO, or SO₂, wherein said ring is optionally fused to a (C6-C10)aryl, (C5-C10)heteroaryl, (C3-C10)cycloalkyl, or a (C3-C10)heterocyclyl;
wherein each occurrence of R is independently substituted with 0-5 R′;
wherein each occurrence of R′ is independently selected from H, halogen, —OH, —R″, —OR″, —NO₂, —NCS, —CN, —CF₃, —OCF₃ and —N(R″)₂;
wherein R″ is H or —(C1-C4)-aliphatic.

3. The compound according to claim 1, wherein Y is —C(R⁴)═.

4. The compound according to claim 3, wherein R⁴ is —H.

5. The compound according to claim 1 or 2, wherein X and the two carbon atoms designated by α and β together form a phenyl ring, optionally substituted with m occurrences of R¹.

6. The compound according to claim 1 or 2, wherein m is an integer selected from 1-4, and at least one R¹ is —OR, wherein R is (C1-C12)-aliphatic- substituted with 0-5 R′.

7. The compound according to claim 6, wherein R is unsubstituted (C1-C4)-aliphatic-.

8. The compound according to claim 7, wherein R is methyl.

9. The compound according to claim 1 or 2, wherein m is an integer selected from 1-4, and at least one R¹ is (C1-C12)-aliphatic- substituted with 0-5 R′.

10. The compound according to claim 9, wherein said at least one R¹ is substituted with at least one —OH.

11. The compound according to claim 1 or 2, wherein m is an integer selected from 1-3, and at least one R¹ is halogen.

12. The compound according to claim 11, wherein said at least one R¹ is Cl— or Br—.

13. The compound according to claim 1 or 2, wherein R² is (C1-C12)-aliphatic-substituted with 0-5 R′.

14. The compound according to claim 13, wherein R² is (C1-C4)-aliphatic-.

15. The compound according to claim 14, wherein R² is methyl, ethyl or isopropyl.

16. The compound according to claim 1 or 2, wherein R³ is (C1-C12)-aliphatic-substituted with 0-5 R′.

17. The compound according to claim 16, wherein R³ is substituted with at least one halogen.

18. The compound according to claim 16, wherein R³ is (C1-C4)-aliphatic-substituted with 0-5 R′.

19. The compound according to claim 1 or 2, wherein R³ is —C(O)OR, wherein R is (C1-C12)-aliphatic- substituted with 0-5 R′.

20. The compound according to claim 19, wherein R is (C1-C4)-aliphatic.

21. The compound according to claim 20, wherein R is methyl or ethyl.

22. The compound according to claim 1 or 2, wherein R³ is —C(O)N(R)₂.

23. The compound according to claim 22, wherein at least one occurrence of R is H.

24. The compound according to claim 22, wherein each R is independently (C1-C4)-aliphatic-.

25. The compound according to claim 24, wherein each R is independently methyl or ethyl.

26. The compound according to claim 22, wherein the two R groups bound to the same nitrogen atom, and the nitrogen atom to which the R groups are bound, together form a 3- to 10-membered aromatic or non-aromatic ring having 0-3 additional heteroatoms independently selected from N, O, S, SO, and SO₂.

27. The compound according to claim 26, wherein said aromatic or non-aromatic ring is a 5-membered or 6-membered ring.

28. The compound according to claim 1 or 2, wherein R³ is (C5-C10)-heteroaryl-, optionally substituted with at least one (C1-C4)-aliphatic-.

29. The compound according to claim 28, wherein R³ is oxadiazole optionally substituted with methyl or ethyl.

30. The compound according to claim 1, wherein
Y is —CH═;
X and the two carbon atoms designated by α and β together form a phenyl ring substituted with 1 substituent selected from halogen and —OR wherein R is
(C1-C4)-alkyl-;
R² is (C1-C4)-alkyl-;
R³ is selected from the group consisting of:
(1) (C1-C4)-alkyl-, substituted with 1 or 2 halogens,
(2) —C(O)OR, wherein R is (C1-C4)-alkyl-,
(3) —C(O)N(R)₂, wherein each R is independently (C1-C4)-alkyl-, or wherein the two R groups together with the nitrogen atom to which they are bound optionally form a 5-membered non-aromatic ring, and
(4) 5-membered heteroaryl- ring having two nitrogen atoms and one oxygen atom, wherein said 5-membered heteroaryl ring is substituted with one (C1-C4)-alkyl-.

31. The compound according to claim 1, wherein said compound is selected from:

| Compound | Structure |
|---|---|
| 2 | 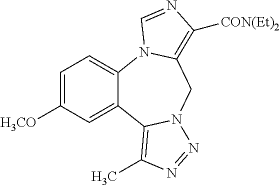 |
| 3 | 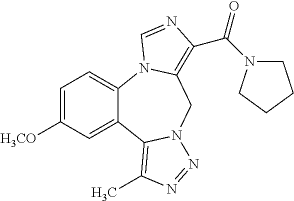 [SMILES: COc1ccc2c(c1)-c1c(C)nnn1Cc1c(C(=O)N3CCCC3)ncn1-2] |
| 4 | 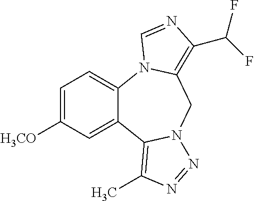 [SMILES: COc1ccc2c(c1)-c1c(C)nnn1Cc1c(C(F)F)ncn1-2] |
| 5 | 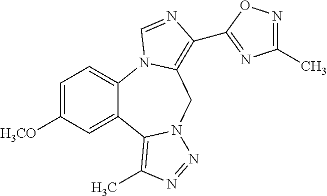 [SMILES: COc1ccc2c(c1)-c1c(C)nnn1Cc1c(-c3nc(C)no3)ncn1-2] |
| 6 | 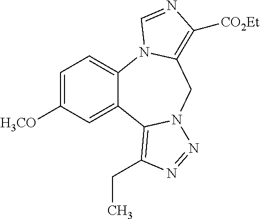 [SMILES: CCOC(=O)c1ncn2c1Cn1nnc(CC)c1-c1cc(OC)ccc1-2] |
| 7 | 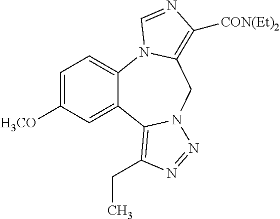 |
| 8 | 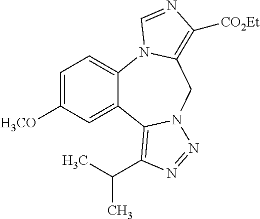 [SMILES: CCOC(=O)c1ncn2c1Cn1nnc(C(C)C)c1-c1cc(OC)ccc1-2] |
| 9 | 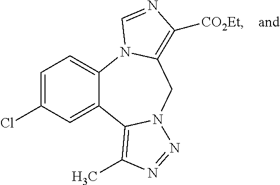 [SMILES: CCOC(=O)c1ncn2c1Cn1nnc(C)c1-c1cc(Cl)ccc1-2 ; "and"] |
| 10 | 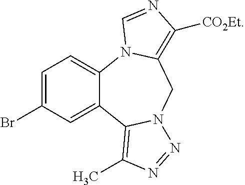 [SMILES: CCOC(=O)c1ncn2c1Cn1nnc(C)c1-c1cc(Br)ccc1-2] |

32. A pharmaceutical composition comprising a compound according to any one of claims 1-31 or a pharmaceutically acceptable salt thereof, in a therapeutically effective amount; and an acceptable carrier, adjuvant or vehicle.

33. A method of treating a central nervous system (CNS) disorder with cognitive impairment in a subject in need thereof, comprising the step of administering a pharmaceutical composition according to claim 32.

34. The method according to claim 33, wherein said CNS disorder with cognitive impairment is age-related cognitive impairment.

35. The method according to claim 34, wherein said age-related cognitive impairment is Age-Associated Memory Impairment (AAMI), Mild Cognitive Impairment (MCI) or Age-Related Cognitive Decline (ARCD).

36. The method according to claim 35, wherein said Age-Related Cognitive Impairment is Mild Cognitive Impairment (MCI).

37. The method according to claim 33, wherein said CNS disorder with cognitive impairment is dementia.

38. The method according to claim 37, wherein said dementia is selected from the group consisting of Alzheimer's disease (AD), vascular dementia, dementia with Lewy bodies and frontotemporal dementia.

39. The method according to claim 38, wherein said dementia is Alzheimer's disease (AD).

40. The method according to claim 33, wherein said CNS disorder with cognitive impairment is schizophrenia.

41. The method according to claim 33, wherein said CNS disorder with cognitive impairment is associated with cancer therapy.

42. The method according to claim 33, wherein said CNS disorder with cognitive impairment is Post Traumatic Stress Disorder (PTSD).
